(19) United States
(12) Patent Application Publication
(10) Pub. No.: US 2022/0283145 A1
(43) Pub. Date: Sep. 8, 2022

(54) METHODS FOR DETECTING, QUANTIFYING AND MONITORING IMMUNE-CELL MEDIATED CYTOTOXICITY OF CELL POPULATIONS COMPROMISING IMMUNE CELLS AND/OR STEM CELLS

(71) Applicant: Fraunhofer-Gesellschaft zur Förderung der angewandten Forschung e.V., München (DE)

(72) Inventors: Stephan FRICKE, Leipzig (DE); Bianca ZÖNNCHEN, Leipzig (DE); Nadja HILGER, Leipzig (DE); Sandy TRETBAR, Leipzig (DE)

(21) Appl. No.: 17/651,587

(22) Filed: Feb. 18, 2022

Related U.S. Application Data
(63) Continuation of application No. PCT/EP2020/073211, filed on Aug. 19, 2020.

(30) Foreign Application Priority Data
Aug. 21, 2019 (EP) 19192847.2

(51) Int. Cl. G01N 33/50; G01N 33/68; G01N 33/574; A61K 35/17; A61K 35/28; A61P 35/00

(52) U.S. Cl. CPC G01N 33/5073; G01N 33/6893; G01N 33/574; A61K 35/17; A61K 35/28; A61P 35/00; G01N 2800/52; G01N 2800/245

(57) ABSTRACT

The present invention relates to an in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells, wherein the isolated cell population is a graft and/or pharmaceutical composition for administration to a patient in need thereof, or is used in the manufacture of said graft and/or pharmaceutical composition as well as to related methods and uses.

The present invention relates to an in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells, wherein the isolated cell population is a graft and/or pharmaceutical composition for administration to a patient in need thereof, or is used in the manufacture of said graft and/or pharmaceutical composition as well as to related methods and uses.

Hematopoietic stem cell transplantations (HSCT) are often the only treatment option after chemotherapy in hemato-oncological diseases (Ghimire et al., 2017). In HSCT the patient is, in addition to the stem cells, also transplanted with immunologically functional T- or other immune cells from a suitable donor in addition to stem cells. In this context, the development of a healthy hematopoietic system in the patient but also the “Graft-versus-Tumor”-effect (GvT-effect), in which the remaining tumor cells are destroyed by the functional cytotoxic cells in the transplant, contributes significantly to the therapeutic success of the treatment (Holtick et al., 2015). The GvT reaction can be negatively influenced by other necessary, supportive therapies, such as immunosuppressants or specific antibodies (Holtick et al., 2015). To date, it has not been possible to directly test and monitor the course of the GvT reaction and the influence of supportive therapies with a specific test.

Accordingly, the main goal of HSCT is the reconstitution of the hematopoietic system, but also the anti-cancer capacity of the donor immune cells against remaining leukemic cells (GvT-effect) is important for the success of the therapy (Holtick et al., 2015).

Besides HSCT, chimeric antigen receptor (CAR) cell-based immunotherapies get more and more into focus of the treatment of hemato-oncological diseases (Castella et al., 2018; Subklewe et al., 2019). Two CAR-T cell therapies are already in the clinics and even more CAR-immune cell products are currently under investigation in clinical trials (Buchholz et al., 2018; U.S. National Library of Medicine, 2019).

Until now, there is no test in clinical use to monitor the functional activity against tumor cells of such transplants or cell products.

Further, there is no test in clinical use to determine, quantify and/or monitor the functional activity of cell products comprising immune and/or stem cells which are either final pharmaceutical products intended for administration into an animal or human, in particular as “Advanced Therapy Medicinal Products” (ATMPs), or are used as starting material or intermediate products for pharmaceutical products intended for administration into an animal or human for therapeutic and/or preventive purposes. Moreover, there is no test in clinical use to determine the effects, such as toxic effects, of supportive therapy regimens used in the context of such pharmaceutical products, e.g. cancer and/or transplantation supportive therapy regimens.

In the prior art, the minimal residual disease after HSCT is examined by different methods, such as fluorescence in situ hybridization (FISH), PCR-based methods, immune phenotyping or sequencing (Kroger et al., 2007; Bader et al., 2009; Lange et al., 2011; Walter et al., 2013; Fu et al., 2014). The selection of suitable methods depends on the type of disease in question (Bader et al., 2018). However, the GvT reaction is not examined directly, but only the extent to which tumor cells are present in the blood, i.e. the success of the therapy is characterized later. The direct investigation of the GvT reaction would be possible by cytotoxicity tests, but these are not yet used in clinical routine. Moreover, there are no methods available to reliably determine, quantify and monitor whether immune and/or stem cells to be transplanted, especially in combination with the intended supportive therapies, such as immunosuppressive drugs, have a functional activity before and after administration to the patient.

The present invention provides methods for detecting, quantifying and monitoring immune-cell mediated cytotoxicity of cell populations comprising immune cells and/or stem cells wherein the cell population is a graft or pharmaceutical composition intended for administration to a patient in need thereof, or is used in the manufacture of said graft and/or pharmaceutical composition.

In one embodiment, the present invention relates to an in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells, wherein the isolated cell population is a graft and/or pharmaceutical composition for administration to a patient in need thereof, or is used in the manufacture of said graft and/or pharmaceutical composition,

comprising the steps of:

(i) optionally enriching and/or purifying immune cells and/or stem cells from the isolated cell population,

(ii) contacting target cells with the isolated cell population or the immune cells and/or stem cells enriched and/or purified in step (i)

(iii) determining the amount and/or concentration of activated caspase-3 in the target cells of (ii), wherein

an amount and/or concentration of said activated caspase-3 above a control, reference or cut-off value is indicative of Graft-versus-Tumor (GvT) activity, compound-mediated toxicity, effectivity, and/or immune-cell mediated cytotoxicity of the isolated cell population.

In one embodiment, the present invention relates to an in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells, wherein the isolated cell population is a graft and/or pharmaceutical composition for administration to a patient in need thereof, or is used in the manufacture of said graft and/or pharmaceutical composition, comprising the steps of:

(i) optionally enriching and/or purifying immune cells and/or stem cells from the isolated cell population,

(ii) contacting target cells with the isolated cell population or the immune cells and/or stem cells enriched and/purified in step (i)

(iii) determining the amount and/or concentration of an apoptosis, necrosis or cell lysis biomarker in the target cells of (ii), wherein an amount and/or concentration of said biomarker above a control, reference or cut-off value is indicative of Graft-versus-Tumor (GvT) activity, compound-mediated toxicity, effectivity, and/or immune-cell mediated cytotoxicity of the isolated cell population.

The advantage of the methods of the invention over previous methods is that the GvT effect of the graft can be measured directly and even before transplantation, and that not the existing tumor load (e.g. any minimal residual disease) in the patient's blood is determined. Further, the methods of the invention allow for reliable and standardized measurement of these effects. These effects are advantageous in the context of assays for use in clinical practice.

The methods of the present invention are based on the mechanism that immune cell-mediated cytotoxicity on tumor cells is detected by a suitable biomarker. In the examples, the biomarker activated caspase-3 was successfully used. The known enzyme caspase-3 in the signaling cascade of programmed cell death (apoptosis) is activated in the tumor cells and detected by flow cytometric analysis (Reed, 2000). In the GvT reaction, tumor cells are specifically recognized by functional cells of the graft and brought into apoptosis, so that caspase-3 is activated in the tumor cells and can be detected. This detection is described in the literature (Jerome et al., 2003), however, it is only used in the prior art for research purposes and not as a relevant test system for clinical applications, for stem cell transplantations and ATMP therapies.

Accordingly, according to one embodiment of the invention, the methods of the invention determine the amount and/or concentration of biomarker activated caspase-3. Thereby, the apoptosis of target cells can be determined.

The methods are therefore useful as test system for grafts and pharmaceutical products comprising cells for providing approval or non-approval, respectively, for administration into a patient, especially for ATMPs.

In the examples, we successfully use a flow cytometry based assay to quantify the T-cell mediated cytotoxic effect of human blood cells or cell transplants against a standardized cell line by detecting activated caspase-3 in apoptotic target cells.

The present invention relates to an in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells. Accordingly, in one alternative, Graft-versus-Tumor (GvT) activity of the isolated cell population is detected or quantified. As Graft-versus-Tumor (GvT) activity results from immune cell-mediated cytotoxicity on tumor cells, an amount and/or concentration of the biomarker above a control, reference or cut-off value is indicative of Graft-versus-Tumor (GvT) activity. The term “cytotoxic” or “cytotoxicity” refers to killing or damaging cells. For example, in case of T cells as immune cells, T cell-mediated cytotoxicity refers to the directed killing of a target cell by a T cell, in particular through the release of granules containing cytotoxic mediators or through the engagement of death receptors.

Further, it is possible that the isolated cell population comprises one or more further compounds, such as pharmaceutically acceptable carriers, adjuvants, solvents, other therapeutically active agents, such agents for supportive therapy, or residual contaminants, such as compounds present in culture media or agents used for freezing or freeze-drying, or particles such as cellular debris or beads for purification. Such one or more further compounds may affect compound-mediated toxicity and/or effectivity of the isolated cell population in vivo. For example, it can be determined whether the one or more further compounds in the cell population exert an inhibitory or stimulatory effect on the immune-cell mediated cytotoxicity of the cell population in vivo, i.e. when administered to a patient or subject.

In the methods of the invention, cell populations comprising immune cells and/or stem cells are analyzed. The immune cells may be T cells (e.g., regulatory T cells, CD4⁺ T cells, CD8⁺ T cells, or gamma-delta T cells), NK cells, invariant NK cells, NKT cells, or stem cells (e.g., mesenchymal stem cells (MSCs) or induced pluripotent stem (iPSC) cells). For example, the cells may be monocytes or granulocytes, e.g., myeloid cells, macrophages, neutrophils, dendritic cells, mast cells, eosinophils, and/or basophils. The cells may be intended for adoptive cell therapy. Thus, the immune cells may be used as immunotherapy, such as to target cancer cells. The stem cells may be inducible pluripotent stem cells (iPS), mesenchymal stem cells, hematopoietic stem cells or embryonal stem cells. In one embodiment, the stem cells are not human embryonal stem cells.

The cell population comprising immune cells and/or stem cells may be obtained from subjects, particularly animal or human subjects, thereby obtaining an isolated cell population or thereby providing an isolated cell population obtained from the patient. Alternatively, the cell population comprising immune cells and/or stem cells may be a cell population comprising genetically modified cells and/or cells derived from cell lines. An “isolated cell population” is understood as an ex vivo or in vitro cell population and/or a cell population spatially separate from an animal or human body. The cell population comprising immune cells and/or stem cells can, for example, be obtained from a patient or subject of interest, such as a subject suspected of having a particular disease or condition, a subject suspected of having a predisposition to a particular disease or condition, a subject who is undergoing therapy for a particular disease or condition, or a subject who is a graft donor. A cell population comprising immune cells and/or stem cells can be collected from any location in which they reside in the subject including, but not limited to, blood, cord blood, spleen, thymus, lymph nodes, and bone marrow. In one preferred embodiment, the cell population comprising immune cells and/or stem cells is obtained from blood. The cell population comprising immune cells and/or stem cells obtained from a subject may be used directly, or they can be stored for a period of time, such as by freezing and/or cultivation.

The isolated cell population is a graft and/or pharmaceutical composition intended for administration to a patient in need thereof, or is used in the manufacture of said graft and/or pharmaceutical composition.

The present method allows for clinical testing of cell populations which are either intended for direct use for administration to a patient, or which are used as starting material or intermediate products for preparing a graft and/or pharmaceutical composition. This in particular applies for cell populations used as grafts, such as hematopoietic stems cells used in HSCT, or so called Advanced Therapy Medicinal Products (ATMP). Such ATMP encompass compositions for gene therapy, compositions for somatic-cell therapy, compositions for tissue-engineered therapy, and combinations thereof (Paul-Ehrlich-Institut, 2010). The isolated cell population comprising immune cells and/or stem cells may be a graft, such as a HSCT graft, or may be an adoptively transferred immune cell or may be a pharmaceutical composition comprising immune cells and/or stem cells.

Accordingly, the method of the invention applies to cell populations which are, as final product, intermediate product or starting material, intended for administration to a patient for therapeutic, preventive or curative purposes. Therefore, the present methods of the invention are in particular useful for the testing of cell populations, which have to fulfill the high requirements of clinical applications.

For example, it is possible to test T cell populations, such as autologous T cell populations which are intended for use as starting material for CAR T cell production. Thereby, the method can be used as test system in order to ensure that only T cell populations are used in the subsequent CAR T cell production which fulfill minimum requirements, such as of cytotoxic potential, as quality control.

Thereby, the production of CAR T cells can be avoided which result in CAR T cells with low therapeutic potential and effectivity. This is important as the production of these products involves high costs. Also, the quality control improves the process for production of effective CAR T cells for patients in need of the therapy. Typically, the patients in need thereof, such as tumor or cancer patients, or patients suffering from autoimmune diseases, and/or rheumatologic diseases, require an effective therapy as soon as possible and/or require a therapy for which effectiveness and safety is ensured.

An preferred example of a therapy for which the methods of the present invention can be applied is the CAR T cell therapy approved as Kymriah® using CAR T cells known as tisagenlecleucel. Kymriah® is an immunocellular therapy containing tisagenlecleucel, autologous T cells genetically modified ex vivo using a lentiviral vector encoding an anti-CD19 chimeric antigen receptor (CAR). The concentration of CAR-positive viable T cells is dependent on indication and patient body weight. The cellular composition and the final cell number varies between individual patient batches. In addition to T cells, NK cells may be present. Typically, the infusion bags contain a total of 1.2×10⁶ to 6×10⁸ CAR-positive viable T cells.

The cells for CAR T cell therapy may be obtained or provided by leukapheresis. Preferably, in cases where autologous T cells are desired, such as in the case of tisagenlecleucel, the cells are obtained and/or provided by leukapheresis of the patient to be treated.

Therefore, in one preferred embodiment, the present invention relates to an in vitro method for detecting and/or quantifying T-cell mediated cytotoxicity of an isolated cell population comprising T cells, wherein the isolated cell population is used in the manufacture of a graft and/or pharmaceutical composition, comprising the steps of:

(i) enriching and/or purifying T cells from the isolated cell population,

(ii) contacting target cells with the T cells enriched and/or purified in step (i)

(iii) determining the amount and/or concentration of activated caspase-3 in the target cells of (ii), wherein

an amount and/or concentration of said activated caspase-3 above a control, reference or cut-off value is indicative of T-cell mediated cytotoxicity of the isolated cell population.

Preferably, the method further comprises the step of preparing a graft and/or pharmaceutical composition comprising T cells in case the amount and/or concentration of said activated caspase-3 above a control, reference or cut-off value.

Preferably, said step of preparing a graft and/or pharmaceutical composition comprises modifying the T cells to comprise an ex vivo genetically engineered T cell receptor (TCR), preferably wherein the ex vivo genetically engineered T cell receptor is a T cell receptor with known antigen-specificity or is a Chimeric Antigen Receptor (CAR) with known antigen-specificity.

As described above, the method is particularly suitable for determining the cytotoxic potential of immune cells and in particular T cells as starting material for CAR immune cell and CAR T cell therapy, such as in the case of tisagenlecleucel. Typically, a portion of the enriched and/or purified cells are tested in the methods of the inventions. Further portion(s) of the enriched and/or purified cells may be stored for subsequent production of CAR immune cells or CAR T-cells, depending on the result of the methods.

Preferably, step (i) of the in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells comprises leukapheresis from blood of a patient.

Blood is a suitable for source of immune cells and in particular T cells.

As described herein, CAR immune cell and in particular CAR T cell therapy is particularly suitable for specifically directing the therapy against an antigen of interest, which is typically an antigen specific for the disease or disorder to be treated.

Preferably, the graft and/or pharmaceutical composition comprises autologous T cells comprising an ex vivo genetically engineered T cell receptor (TCR), preferably wherein the ex vivo genetically engineered T cell receptor is a T cell receptor with known antigen-specificity or is a Chimeric Antigen Receptor (CAR) with known antigen-specificity.

Preferably, the ex vivo genetically engineered T cell receptor has antigen-specificity for a target selected from the group consisting of CD19, CD20, CD123, mesothelin, CD4, CD5, CD38, CD47, CLL-1, CD33, CD200, CS1, BAFF-R, ROR-1, CD99, HSP70, and BCMA.

For example, tisagenlecleucel comprises a Chimeric Antigen Receptor with specificity for antigen CD19.

As used herein, the term “patient” or “subject” refers to a living mammalian organism, such as a human, monkey, cow, sheep, horse, goat, dog, cat, mouse, rat, guinea pig, or transgenic species thereof. In certain embodiments, the patient or subject is a primate, in particular a human. Examples of human patients are adults, juveniles, infants and fetuses.

According to step (i), the immune cells are optionally enriched and/or purified from the cell population. For example, when the cell population is obtained from a sample of a patient or subject, the cell population may be from any tissue where they reside including blood (including blood collected by blood banks or cord blood banks), spleen, bone marrow, tissues removed and/or exposed during surgical procedures, and tissues obtained via biopsy procedures. Tissues/organs from which the immune cells are optionally enriched and/or purified may be isolated from both living and non-living subjects, wherein the non-living subjects are organ donors. In particular embodiments, the cell population is blood or is from blood, such as peripheral blood or cord blood. By “enriched” is meant a composition comprising cells present in a greater percentage of total cells than is found in the isolated cell population. For example, the immune cells and/or stem cells may be enriched or purified, for example enriched by a factor 2-, 5-, 10, 50-, 100- or 1000-fold (immune cells and/or stem cells/total cells) and/or purified, such as purified to 20%, 30%, 40%, 50%, 60%, 70%, 80%, 90, 91%, 92%, 93%, 94%, 95%, 96%, 97%, 98%, 99% or 100% (immune cells and/or stem cells/total cells) purity.

Enriching and/or purifying may be performed by methods known in the art, such as cell sorting, including FACS, centrifugation, such as density gradient centrifugation, lysis of erythrocytes in case of blood, or by apheresis devices (Amos et al., 2012; Dagur and McCoy, 2015).

Step (ii) of the method of the invention relates to contacting target cells with the isolated cell population or the immune cells and/or stem cells enriched and/purified in step (i). The target cells are any suitable type of cell, which can be brought into apoptosis, necrosis or cell lysis by specific interaction with the optionally enriched and/or purified immune cell and/or stem cells. In particular, the target cells are any suitable type of cell, which can be brought into apoptosis by specific interaction with the optionally enriched and/or purified immune cell and/or stem cells. For example, in the case of cytotoxic T cells and/or hematopoietic stem cell grafts, the target cell may be for example a tumor cell, a target cell population comprising one single target cell type or a target cell population comprising 2 or more different cell types, or an infected cell, such as a cell infected with a virus. The cells may be brought into lysis for example by the action of perforin, which may be released by immune cells, such as CTLs or NK cells (Bolitho et al., 2007). The cells may be brought into apoptosis for example by engagement of Fas with FasL, which allows for recruitment of the death-induced signaling complex (DISC) (Caulfield and Lathem, 2014). The Fas-associated death domain (FADD) translocates with the DISC, allowing recruitment of procaspases 8 and 10. These caspases then activate the effector caspases 3, 6, and 7, leading to cleavage of death substrates such as lamin A, lamin B1, lamin B2, PARP (poly ADP ribose polymerase), and DNA-PKcs (DNA-activated protein kinase) (Olsson and Zhivotovsky, 2011). Moreover, the cells may be brought into apoptosis for example by the cytotoxins perforin, granzymes, and granulysin released by the immune cells and/or stem cells. Through the action of perforin, granzymes enter the cytoplasm of the target cell and their serine protease function triggers the caspase cascade, which is a series of cysteine proteases that eventually lead to apoptosis (programmed cell death) (Chowdhury and Lieberman, 2008). Moreover, the cells may be brought into necrosis by for example by the production of ROS (reactive oxygen species) or RNS (reactive nitrogen species) of the immune cells, which in turn, leads to DNA damage and necrosis (Muralidharan and Mandrekar, 2013).

In step (iii), the amount and/or concentration of an apoptosis, necrosis or cell lysis biomarker in the target cells is determined. In particular, in step (iii), the amount and/or concentration of an apoptosis biomarker in the target cells is determined. A biomarker is a characteristic objectively measured and evaluated to indicate normal or pathogenic biological processes or pharmacologic response. A biomarker may for example be a nucleic acid or protein, a carbohydrate, or inorganic ions or salts, such as Ca²⁺ or release of detectable compounds from cells, such as radioactive chromium ions/salts. Such biomarkers for apoptosis, necrosis or cell lysis are known in the art. For example, suitable biomarkers for apoptosis include an activated caspase, preferably activated caspase-3, activated caspase-6, and/or activated caspase-7, cleaved poly(ADP-ribose) polymerase and Ca²⁺(Duriez and Shah, 1997; Pinton et al., 2008; Olsson and Zhivotovsky, 2011). For example, a suitable biomarker for cell lysis is ⁵¹Cr. For example, the ⁵¹Cr release assay known in the art may be used, wherein internalized ⁵¹Cr is released to greater extent from apoptotic and/or lysed cells (Brunner et al., 1968).

In one embodiment of the present invention, the biomarker is activated caspase-3.

Caspases (cysteine-aspartic proteases, cysteine aspartases or cysteine-dependent aspartate-directed proteases) are a family of protease enzymes playing essential roles in programmed cell death, including apoptosis, pyroptosis and necroptosis, and inflammation. They are named caspases due to their specific cysteine protease activity—a cysteine in its active site nucleophilically attacks and cleaves a target protein only after an aspartic acid residue. Caspases, which function as initiator in apoptosis, are known in the art and include caspase 2, caspase 8, caspase 9, and caspase 10. Caspase-8 is also required for the inhibition of another form of programmed cell death called necroptosis. Caspases, which function as executioner in apoptosis, are known in the art and include caspase 3, caspase 6, and caspase 7 (Olsson and Zhivotovsky, 2011). Caspases, which exert an inflammatory effect in pyroptosis, are known in the art and include caspase 1, caspase 4, caspase 5, caspase 11 and caspase 12 (Fink and Cookson, 2005). Caspases are synthesized as inactive zymogens (pro-caspases) that are only activated following an appropriate stimulus (Reed, 2000). This post-translational level of control allows rapid and tight regulation of the enzyme. Activation involves dimerization and often oligomerization of pro-caspases, followed by cleavage into a small subunit and large subunit. The large and small subunit associate with each other to form an active heterodimer caspase (Reed, 2000). The active enzyme often exists as a heterotetramer in the biological environment, where a pro-caspase dimer is cleaved together to form a heterotetramer. For example, activated caspase-3 comprises caspase-3 cleaved by initiator caspases.

It is within the skills of the practitioner to choose an appropriate control sample/population/cohort/group and a control or reference value for the biomarker established therein. It is clear to the skilled artisan, that the absolute marker values established in a control will be dependent on the assay used. Preferably, samples from well-characterized cell population(s) are used to establish a control or reference value.

The determining of the amount and/or concentration of an apoptosis, necrosis or cell lysis biomarker in the target cells is performed by methods known in the art, depending on the biomarker. For example, in the case of a protein biomarker, such as an activated caspase including activated caspase-3, activated caspase-6, or activated caspase-7, or cleaved poly(ADP-ribose) polymerase, the protein may be detected by flow cytometry or an immunoassay, such as an ELISA or immunohistochemical assay using a binding agent, such as an antibody, which specifically binds to the biomarker. Preferred immunoassays, which can be used in the present invention, include Enzyme Linked Immunosorbent Assay (ELISA) (Engvall and Perlmann, 1971), electrochemical assay (ECL), electrochemi-luminescent immunoassay (ECLIA) (Forster et al., 2009), radioimmunoassay (RIA) (Blake and Ban, 2014) or ultra-sensitive single molecule array assay (Simoa) (Rissin et al., 2010).

Accordingly, in one embodiment, the protein activated caspase-3 may be detected by flow cytometry or an immunoassay, such as an ELISA or immunohistochemical assay using a binding agent, such as an antibody, which specifically binds to the biomarker activated caspase-3.

Alternatively, in case of a nucleic acid biomarker, hybridization of suitable nucleic acid probes, such as DNA or RNA or nucleic acid derivatives such as PNA or LNA may be used (Wang et al., 1997; Petersen and Wengel, 2003). For example, an mRNA biomarker can be detected by any technique known in the art. These include Northern blot analysis, reverse transcriptase-PCR amplification (RT-PCR), microarray analysis and RNase protection (ThermoFisher Scientific, 2019a).

For example, mRNA in target cells can be measured in a Northern blot assay. Here, tissue RNA is fractionated by electrophoresis, fixed to a solid membrane support, such as nitrocellulose or nylon, and hybridized to a probe capable of selectively hybridizing with biomarker mRNA in the target cell sample (Alwine et al., 1977).

In another embodiment, the biomarker mRNA is amplified and quantitatively assayed. The polymerase chain reaction (PCR) procedure can be used to amplify specific nucleic acid sequences through a series of iterative steps including denaturation, annealing of oligonucleotide primers designed according to the biomarker mRNA sequence, and extension of the primers with DNA polymerase (Mullis et al., 1986). In reverse transcriptase-PCR (RT-PCR), this procedure is preceded by a reverse transcription step to allow a large amplification of the number of copies of mRNA (Mo et al., 2012).

Quantitation of RT-PCR products can be done while the reaction products are building up exponentially, and can generate diagnostically useful clinical data. In one embodiment, the measurement is carried out by reference to one or more housekeeping genes which are also amplified by RT-PCR. Quantitation of a RT-PCR product may be undertaken, for example, by gel electrophoresis visual inspection or image analysis, HPLC or by use of fluorescent detection methods (ThermoFisher Scientific, 2019b).

In one preferred embodiment, the amount and/or concentration determined for the biomarker in a control cell population is for example used to establish a cut-off value or a reference range. A value above such cut-off value or out-side the reference range and its higher end is considered as apoptosis, necrosis or cell lysis. In one embodiment, a fixed cut-off value is established. Such cut-off value is chosen to match the question of interest. A suitable cut-off value may be chosen depending on the sensitivity and specificity desired.

In another embodiment, the present invention relates to an in vitro method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered, wherein the cell population is a graft and/or pharmaceutical composition, comprising the steps of:

(0i) obtaining a sample from the patient, preferably wherein the sample is a blood sample,

(i) enriching and/or purifying immune cells from the sample,

(ii) contacting target cells with the immune cells and/or stem cells enriched and/or purified in step (i),

(iii) determining the amount and/or concentration of an apoptosis, necrosis or cell lysis biomarker in the target cells of (ii), wherein

an amount and/or concentration of said biomarker above a control, reference or cut-off value is indicative of Graft-versus-Tumor (GvT) activity, effectivity, compound-mediated toxicity and/or immune-cell mediated cytotoxicity of the cell population in the patient.

In another embodiment, the present invention relates to an in vitro method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered, wherein the cell population is a graft and/or pharmaceutical composition, comprising the steps of:

(0i) providing a sample obtained from the patient,

(i) enriching and/or purifying immune cells from the sample,

(ii) contacting target cells with the immune cells and/or stem cells enriched and/or purified in step (i),

(iii) determining the amount and/or concentration of activated caspase-3 in the target cells of (ii), wherein

an amount and/or concentration of said activated caspase-3 above a control, reference or cut-off value is indicative of Graft-versus-Tumor (GvT) activity, effectivity, compound-mediated toxicity and/or immune-cell mediated cytotoxicity of the cell population in the patient.

Preferably, the sample is a blood sample.

In this embodiment of the present invention, the method is an in vitro method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered.

The method allows for determining and/or quantifying Graft-versus-Tumor (GvT) activity, effectivity, compound-mediated toxicity, and/or immune-cell mediated cytotoxicity of a cell population after administration to the patient. For example, the cell population comprising immune cells and/or stem cells may be a graft, such as a HSCT graft, or may be an adoptively transferred immune cell or may be a pharmaceutical composition comprising immune cells and/or stem cells.

This method enables the in vitro determination and quantification of whether the administered cell population comprising immune cells and/or stem cells exhibits Graft-versus-Tumor (GvT) activity and/or immune-cell mediated cytotoxicity in vivo in the body of the patient. In case of a graft or any other cell population comprising immune cells and/or stem cells for which a Graft-versus-Tumor (GvT) activity and/or immune-cell mediated cytotoxicity is desired, such as an adoptively transferred immune cell, the method is in particular useful for determining the effectivity of the cell population in the body of the patient.

Further, it is possible that the cell population, when administered to the patient, comprises one or more further compounds, such as pharmaceutically acceptable carriers, adjuvants, solvents, other therapeutically active agents, such agents for supportive therapy, or residual contaminants, such as compounds present in culture media or agents used for freezing or freeze-drying, or particles such as cellular debris or beads for purification. Such one or more further compounds may affect compound-mediated toxicity and/or effectivity of the cell population in the body of the patient after administered. Moreover, the patient may in addition obtain further therapies, such as therapies for confounding diseases or supportive therapies. Such therapies may be administered together, or temporally and/or spatially separate from the cell population. Such therapies may also affect compound-mediated toxicity and/or effectivity of the cell population in the body of the patient after administered. In particular, it can be determined with the present method of the invention, whether one or more further compounds and/or therapies exert(s) an inhibitory or stimulatory effect on the immune-cell mediated cytotoxicity of the cell population or otherwise exert(s) a compound-mediated toxicity on the cell population.

A supportive therapy is understood as a treatment regimen designed to improve, reinforce, or sustain a patient's physiological well-being or psychological self-esteem and self-reliance. A supportive therapy in cancer or cancer supportive therapy comprises, for example, palliative care, treatment with one or more antiemetic, in particular to treat nausea and vomiting, treatment with one or more bone marrow growth factor, in particular to reduce myelosuppression, treatment with enteral or parenteral feeding, treatment with one or more antiviral agent, treatment with one or more antibacterial agent, treatment with one or more antimycotic agent, treatment with one or more analgetic agent (Leitlinienprogramm Onkologie, 2017). A supportive therapy in transplantation or transplantation supportive therapy, such as HSCT, comprises for example, the administration of one or more immunosuppressants. Useful immunosuppressants are known in the art and include calcineurin inhibitors and steroids, in particular corticosteroids. Examples of transplantation supportive therapies, such as HSCT, comprises calcineurin inhibitors, such as tacrolimus or cyclosporine A, corticosteroids such as prednisolone, or methylprednisolone, cytostatic or cytotoxic agents, such as methotrexate, antithymocyte globulin, antibodies such as anti-CD4 antibody, anti-interleukin-2 (anti-IL-2) receptor antibody, anti-tumor necrosis factor a (anti-TNF-α) antibody, and anti-CD52 antibody, a statin such as pentostatin, an antibiotic, or combinations thereof (Zeiser et al., 2019). The antibodies may be for ex vivo incubation with the cell population, prior to administration, or for in vivo administration. For example, an anti-CD4 antibody may be used for ex vivo incubation with the cell population, prior to administration, or for in vivo administration to a patient.

In the examples, the influence of Palixizumab®, which is an anti-CD4 antibody for supportive cancer therapy, on the cytotoxicity of PBMCs is successfully detected and quantified.

In step (0i), a sample is obtained from the patient. The sample may be any sample comprising at least one immune cell and/or stem cell, or cell derived from said immune cell and/or stem cell, of the cell population administered to the patient. A “cell derived from” said immune cell and/or stem cell may be a cell derived from the administered cell by cell division and/or by differentiation. A “sample” refers to a biological sample obtained for the purpose of evaluation in vitro. It comprises material which can be specifically related to the individual and from which specific information about the individual can be determined, calculated or inferred. A sample can be composed in whole or in part of biological material from the patient or subject, such as for example a blood sample. The sample may preferably comprise a body fluid. Exemplary samples include blood, bronchoalveolar lavage,

PBMC, urine, saliva, whole blood, or cerebrospinal fluid (CSF). The sample may be taken from the individual and used immediately or processed before step (i).

In a preferred embodiment, the sample is a blood sample. A blood sample may be obtained easily and sufficient amount of blood can be obtained from the patient.

The use of blood as preferred sample allows for particularly simple and reliable implementation of the methods of the invention into clinical practice and routine.

Steps (i), (ii) and (iii) of the method of the invention refer to:

(i) enriching and/or purifying immune cells from the sample,

(ii) contacting target cells with the immune cells and/or stem cells enriched and/or purified in step (i),

(iii) determining the amount and/or concentration of an apoptosis, necrosis or cell lysis biomarker in the target cells of (ii).

For these steps, the same embodiments apply as for the method of the invention above.

In the method of the invention, an amount and/or concentration of said biomarker above a control, reference or cut-off value is indicative of Graft-versus-Tumor (GvT) activity, effectivity, compound-mediated toxicity and/or immune-cell mediated cytotoxicity of the cell population in the patient.

Accordingly, additional therapies or amendments to existing therapies, such as supportive therapies, may be considered for the patient in case an amount and/or concentration of said biomarker below a control, reference or cut-off value is indicative of lack of, or insufficient, Graft-versus-Tumor (GvT) activity, effectivity and/or immune-cell mediated cytotoxicity of the cell population in the patient.

In yet another embodiment, the present invention relates to an in vitro method for monitoring Graft-versus-Tumor (GvT) activity effectivity, compound-mediated toxicity and/or immune-cell mediated cytotoxicity of a cell population comprising immune cells and/or stem cells in a patient to whom the cell population was previously administered, wherein the isolated cell population is a graft and/or pharmaceutical composition, comprising the steps of:

(A)

(a) performing the method for detecting and/or quantifying Graft-versus-Tumor (GvT) activity, compound-mediated toxicity, effectivity and/or immune-cell mediated cytotoxicity of an isolated cell population comprising immune cells and/or stem cells described herein, and

(b) performing the method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered described herein at 1 or more time point(s) after the cell population was administered to the patient, or

(B)

(a) optionally performing the method for detecting and/or quantifying Graft-versus-Tumor (GvT) activity, compound-mediated toxicity, effectivity and/or immune-cell mediated cytotoxicity of an isolated cell population comprising immune cells and/or stem cells described herein, and

(b) performing the method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered described herein at 2 or more time points after the cell population was administered to the patient,

wherein an increase in amount and/or concentration of said biomarker in the target cells at a later time point is indicative of an increase in Graft-versus-Tumor (GvT) activity, effectivity, compound-mediated toxicity and/or immune-cell mediated cytotoxicity of the cell population in the patient, and wherein a decrease in amount and/or concentration of said biomarker in the target cells at a later time point is indicative of a decrease in Graft-versus-Tumor (GvT) activity, effectivity, compound-mediated toxicity, and/or immune-cell mediated cytotoxicity of the cell population in the patient.

In yet another embodiment, the present invention relates to an in vitro method for monitoring Graft-versus-Tumor (GvT) activity effectivity, compound-mediated toxicity and/or immune-cell mediated cytotoxicity of a cell population comprising immune cells and/or stem cells in a patient to whom the cell population was previously administered, wherein the isolated cell population is a graft and/or pharmaceutical composition, comprising the steps of:

(A)

(a) performing the method for detecting and/or quantifying Graft-versus-Tumor (GvT) activity, compound-mediated toxicity, effectivity and/or immune-cell mediated cytotoxicity of an isolated cell population comprising immune cells and/or stem cells described herein, and

(b) performing the method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered described herein at 1 or more time point(s) after the cell population was administered to the patient, or

(B)

(a) optionally performing the method for detecting and/or quantifying Graft-versus-Tumor (GvT) activity, compound-mediated toxicity, effectivity and/or immune-cell mediated cytotoxicity of an isolated cell population comprising immune cells and/or stem cells described herein, and

(b) performing the method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered described herein at 2 or more time points after the cell population was administered to the patient,

wherein an increase in amount and/or concentration of said activated caspase-3 in the target cells at a later time point is indicative of an increase in Graft-versus-Tumor (GvT) activity, effectivity, compound-mediated toxicity and/or immune-cell mediated cytotoxicity of the cell population in the patient, and wherein a decrease in amount and/or concentration of said activated caspase-3 in the target cells at a later time point is indicative of a decrease in Graft-versus-Tumor (GvT) activity, effectivity, compound-mediated toxicity, and/or immune-cell mediated cytotoxicity of the cell population in the patient.

The method allows for monitoring a patient to whom the cell population comprising immune cells and/or stem cells was administered.

For example, it is possible pursuant to alternative A) to detect and/or quantify Graft-versus-Tumor (GvT) activity, compound-mediated toxicity, effectivity and/or immune-cell mediated cytotoxicity of an isolated cell population comprising immune cells and/or stem cells as described above. A sample of the isolated cell population intended for administration to the patient may be retained for use in the method of the invention prior to the administration to the patient. The result of the method may be available prior to the administration of the cell population to the patient or after the administration of the cell population to the patient.

At 1 or more time point(s), after the cell population was administered to the patient, the method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered described herein is performed.

Thereby, the Graft-versus-Tumor (GvT) activity, effectivity, compound-mediated toxicity, and/or immune-cell mediated cytotoxicity of a cell population prior to and after administration can be compared and thereby monitored. Any suitable time point after administration to the patient may be chosen. For example, the time point may be 1, 3, 6, 12, 15, 18, 24 or more hours, 1, 2, 3, 4, 5, 6, 7 or more days, 1, 2, 3, 4, 5, 6, 7, 8, 9, 10 or more weeks or 1, 2, 3, 4, 5, 6, 7, 8, 9, 10 or more months after the administration of the cell population comprising immune cells and/or stem cells to the patient. Further, the method may be performed at more than 1 time point after the cell population was administered to the patient. In such preferred embodiment, the method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered described herein is repeated one or more times. Accordingly, the Graft-versus-Tumor (GvT) activity, effectivity, compound-mediated toxicity, and/or immune-cell mediated cytotoxicity of the cell population may additionally be monitored over time after administration of the cell population to the patient.

The method can be repeated 1, 2, 3, 4, 5, 6, 7, 8, 9, 10 or more times. The method may be performed at 2, 3, 4, 5, 6, 7, 8, 9, 10, 11 or more time point after the cell population was administered to the patient.

Further, it is possible pursuant to alternative B) to monitor Graft-versus-Tumor (GvT) activity, effectivity, compound-mediated toxicity, and/or immune-cell mediated cytotoxicity of a cell population comprising immune cells and/or stem cells in a patient to whom the cell population was previously administered. In this alternative embodiment, the method is performed at 2 or more time points after the cell population was administered to the patient. Optionally, the method for detecting and/or quantifying Graft-versus-Tumor (GvT) activity, compound-mediated toxicity, effectivity and/or immune-cell mediated cytotoxicity of an isolated cell population comprising immune cells and/or stem cells described herein is performed as first step.

The method allows monitoring the cell population previously administered to the patient over time.

The method can be repeated 1, 2, 3, 4, 5, 6, 7, 8, 9, 10 or more times. The method may be performed at 2, 3, 4, 5, 6, 7, 8, 9, 10, 11 or more time points after the cell population was administered to the patient.

Thereby, a time course can be established, which allows for monitoring the Graft-versus-Tumor (GvT) activity, effectivity, compound-mediated toxicity, and/or immune-cell mediated cytotoxicity of the cell population in the patients. The time intervals may differ, and, for example, may be a time interval between 1 day and 20 years, such as between 1 week, 1 month and 1, 2, 3, 4, 5, 6, 7, 8, 9, or 10 years. Also, the time intervals may differ in case the method is repeated 2, 3, 4, 5, 6, 7, 8, 9, 10 or more different times.

For example, the control may be isolated cell population(s), or a cell population(s) obtained from a healthy control individual or a healthy control cohort, or may be from the same individual at a respective earlier time point, such as from the same individual at the first time point or the isolated cell population prior to administration.

In the method of the invention, an increase in amount and/or concentration of said biomarker in the target cells at a later time point is indicative of an increase in Graft-versus-Tumor (GvT) activity, effectivity, compound-mediated toxicity and/or immune-cell mediated cytotoxicity of the cell population in the patient, and a decrease in amount and/or concentration of said biomarker in the target cells of (ii) at a later time point is indicative of a decrease in Graft-versus-Tumor (GvT) activity, effectivity, compound-mediated toxicity, and/or immune-cell mediated cytotoxicity of the cell population in the patient.

For example, an increase in amount and/or concentration of the biomarker in the target cells at later time points indicates that the Graft-versus-Tumor (GvT) activity, effectivity, compound-mediated toxicity and/or immune-cell mediated cytotoxicity of the cell population comprising immune cells and/or stem cells in the patient increases.

In one preferred embodiment, the patient to whom a cell population comprising immune cells and/or stem cell was previously administered suffers from an immune disease or disorder and is treated with at least supportive therapy. It is possible with the method of the invention to identify patients in which the supportive therapy affects, for example increases or decreases the Graft-versus-Tumor (GvT) activity and/or immune-cell mediated cytotoxicity of the cell population and thereby affects effectivity and/or compound-mediated toxicity. In a more preferred embodiment, the patient to whom a cell population comprising immune cells and/or stem cell was previously administered suffers from cancer and is treated with at least one cancer supportive therapy and/or has obtained a graft, and is treated with at least one transplantation supportive therapy. It is possible with the method of the invention to identify patients in which change in the supportive therapy, in particular the cancer and/or transplantation supportive therapy, is applicable.

Therefore, in one preferred embodiment of the methods of the present invention, the patient is treated with at least one cancer and/or transplantation supportive therapy, and the patient is identified as a patient for which a change in the cancer and/or transplantation supportive therapy is applicable

(i) in case an amount and/or concentration of said biomarker below a control, reference or cut-off value is determined pursuant to a method of the invention described herein, and/or in case a decrease in amount and/or concentration of said biomarker in the target cells at least one later time point is determined pursuant to a method of the invention described herein, or

(ii) in case an amount and/or concentration of said biomarker above a control, reference or cut-off value is determined pursuant to a method of the invention described herein, and/or

in case an increase in amount and/or concentration of said biomarker in the target cells at least one later time point is determined pursuant to a method of the invention described herein.

Therefore, in one preferred embodiment of the methods of the invention, the patient is treated with at least one cancer and/or transplantation supportive therapy, and wherein the patient is identified as a patient for which a change in the cancer and/or transplantation supportive therapy is applicable

(i) in case an amount and/or concentration of said activated caspase-3 below a control, reference or cut-off value is determined pursuant to a method of the invention described herein, and/or

in case a decrease in amount and/or concentration of said activated caspase-3 in the target cells at least one later time point is determined pursuant to a method of the invention described herein, or

(ii) in case an amount and/or concentration of said activated caspase-3 above a control, reference or cut-off value is determined pursuant to a method of the invention described herein, and/or

in case an increase in amount and/or concentration of said activated caspase-3 in the target cells at least one later time point is determined pursuant to a method of the invention described herein.

For example, it may be desirable to maintain a high level of immune-mediated cytotoxicity and/or GvT activity of the cell population in the patient. In case a decrease in the amount and/or concentration of the biomarker in the target cells is determined at a later time point, or an amount and/or concentration below a control is determined, the patient is identified to be a patient for which a change in the supportive therapy is applicable which results in an increased immune cell function in the patient.

It may also be possible that it is desired that the level of immune-mediated cytotoxicity and/or GvT activity of the cell population in the patient is low. In case an increase in the amount and/or concentration of the biomarker in the target cells is determined at a later time point, or an amount and/or concentration above a control is determined, the patient is identified to be a patient for which a change in the supportive therapy is applicable which results in a decreased immune cell function in the patient.

For example, the change in supportive therapy which results in a decreased or increased immune cell function in the patient may be changing any therapy with immunosuppressant(s). For example, the patient did not obtain any immunosuppressant, and the change is the administration of immunosuppressant(s). Such change will result in a decreased immune cell function in the patient. For example, the change is the administration of different immunosuppressant(s), exerting a lower or higher immunosuppressive activity, respectively, and/or changing the dose and/or frequency of the previously administered immunosuppressant(s). For example, the patient previously obtained immunosuppressant(s), and the change is that no immunosuppressant(s) is/are administered.

Accordingly, in one preferred embodiment, in case of (i), the change in the cancer and/or transplantation supportive therapy results in an increased immune cell function in the patient, or in case of (ii), the change in the cancer and/or transplantation supportive therapy results in a decreased immune cell function in the patient.

In yet another preferred embodiment, the cancer and/or transplantation supportive therapy is administration of one or more immunosuppressant(s). Further, in yet another preferred embodiment, a change in the cancer and/or transplantation supportive therapy resulting in an increased immune cell function in the patient is administering one or more different immunosuppressant(s) and/or administering a lower dose or frequency of one or more different immunosuppressant(s), or change in the cancer and/or transplantation supportive therapy resulting in a decreased immune cell function in the patient is administering one or more different immunosuppressant(s) and/or administering a higher dose or frequency of one or more different immunosuppressant(s).

Determining the amount and/or concentration of an apoptosis, necrosis or cell lysis biomarker in the target cells pursuant to the methods of the invention may be performed by flow cytometry, a bioluminescence assay, a ⁵¹Cr release assay, a calcein release assay, or an europium assay or any other comparable assay suitable for determining the amount and/or concentration of said biomarker. Such assays are well-known in the art and include ELISA, RT-PCR, Western blot, Northern blot, FISH, Southern blot, IHC and RIA. For example, the calcein-release is disclosed in (Lichtenfels et al., 1994). A ⁵¹Cr release assay is for example disclosed in (Wallace et al., 2004). Further, an europium assay is known and is disclosed e.g. in (Vikström S., Lähde T. and Blomberg K.) An ATP bioluminescence assay is for example disclosed in (Karimi et al., 2014).

Therefore, in one preferred embodiment, the amount and/or concentration of said biomarker is determined by flow cytometry, a bioluminescence assay, a ⁵¹Cr release assay, a calcein release assay, or an europium assay.

Determining the amount and/or concentration of activated caspase-3 was successfully determined by flow cytometry in the examples.

Therefore, in one preferred embodiment, the amount and/or concentration of said activated caspase-3 is determined by flow cytometry.

For example, in case the biomarker is a protein, or carbohydrate, an immunoassay may be employed using a binding agent specifically binding to the biomarker, wherein the binding agent comprises a detectable label. A specific binding agent is, e.g., a receptor for the biomarker or an antibody to the biomarker or a nucleic acid complementary to the nucleic acid relating to the biomarker (e.g. a nucleic acid complementary to a biomarker's mRNA or relevant part thereof).

A specific binding agent has preferably at least an affinity of 10⁷l/mol for its corresponding target molecule. The specific binding agent preferably has an affinity of 10⁸ l/mol or even more preferred of 10⁹ l/mol for its target molecule (Bishop et al., 2013). As the skilled artisan will appreciate, the term specific is used to indicate that other biomolecules present in the sample do not significantly bind to the binding agent specific for the marker. Preferably, the level of binding to a biomolecule other than the target molecule results in a binding affinity which is only 10% or less, more preferably only 5% or less of the affinity to the target molecule, respectively. A preferred specific binding agent will fulfill both the above minimum criteria for affinity as well as for specificity.

In the examples, activated caspase-3 was successfully used as biomarker for apoptosis (Reed, 2000). Activated caspase-3 was determined by flow cytometry using an antibody specifically binding to activated caspase-3, wherein the antibody was conjugated to a fluorescent label. In the example, the proportion of target cells labelled with the binding agent specifically binding to the biomarker is determined to be indicative of apoptosis of the target cells.

Accordingly, in yet another preferred embodiment, the amount and/or concentration of said activated caspase-3 in step (iii) is determined by flow cytometry and/or comprises contacting the target cells with a binding agent specifically binding to the activated caspase-3, wherein the binding agent comprises a detectable label. Preferably, the proportion of target cells labelled with the binding agent specifically binding to the activated caspase-3 is determined, and/or wherein the detectable label is a fluorescent label, and/or wherein the amount and/or concentration of the activated caspase-3 is indicative of apoptosis of the target cells.

Accordingly, in yet another preferred embodiment, the amount and/or concentration of said biomarker in step (iii) is determined by flow cytometry and/or comprises contacting the target cells with a binding agent specifically binding to the biomarker, wherein the binding agent comprises a detectable label. Preferably, the proportion of target cells labelled with the binding agent specifically binding to the biomarker is determined, and/or wherein the detectable label is a fluorescent label, and/or wherein the amount and/or concentration of the biomarker is indicative of apoptosis of the target cells.

A detectable label or moiety may be directly or indirectly detectable. For example, an enzyme, dye, radionuclide, luminescent group, fluorescent group or biotin, or the like may be used (Blake and Ban, 2014; Lakshmipriya et al., 2016). Any reporter moiety or label could be used with the methods disclosed herein as long as the signal of such is directly related or proportional to the quantity of binding agent. Any unreacted material may be washed away. For radioactive groups, scintillation counting or autoradiographic methods are generally appropriate. Antibody-enzyme conjugates can be prepared using a variety of coupling techniques (Prasse et al., 2014). Spectroscopic methods can be used to detect dyes (including, for example, colorimetric products of enzyme reactions), luminescent groups and fluorescent groups (R&D Systems). Biotin can be detected using avidin or streptavidin, coupled to a different reporter group (commonly a radioactive or fluorescent group or an enzyme). Enzyme reporter groups can generally be detected by the addition of substrate (generally for a specific period of time), followed by spectroscopic, spectrophotometric or other analysis of the reaction products. Standards and standard additions can be used to determine the level of antigen in a sample, using well known techniques summarized in (Sittampalam et al., 2004).

The term “antibodies” includes polyclonal antibodies, monoclonal antibodies, antigen-binding fragments thereof, as well as any naturally occurring or recombinantly produced binding partners, which are molecules that specifically bind a biomarker. The term “antigen-binding fragments” of an antibody refers to molecules which possess the ability to bind to an antigen in a similar fashion as an antibody but which is smaller in size than a complete antibody molecule. Exemplified, two “antigen binding fragments” of an antibody are obtained by papain digestion which produces three fragments, namely two identical fragments, called “Fab fragments” (also referred to as “Fab portion” or “Fab region”) each with a single antigen binding site, and a residual “Fc fragment” (also referred to as “Fc portion” or “Fc region”) whose name reflects its ability to crystallize readily (Janeway, 2001). Further antigen-binding fragments include “Fab' fragment” which refer to a Fab fragment additionally comprise the hinge region of an Ig molecule, and “F(ab′)₂ fragments” which are understood to comprise two Fab′ fragments being either chemically linked or connected via a disulfide bond. Further encompassed are “Nanobodies” which only comprise a single VH domain, “single chain Fv (scFv)” fragments comprise the heavy chain variable domain joined via a short linker peptide to the light chain variable domain, divalent single-chain variable fragments (di-scFvs) which can be engineered by linking two scFvs (scFvA-scFvB) (Alam et al., 2018). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies” (Holliger et al., 1993). Still shorter linkers (one or two amino acids) between a VH and VL domain lead to the formation of monospecific trimers, so-called “triabodies” or “tribodies” (Iliades et al., 1997). Bispecific diabodies are formed by expressing two chains with the arrangement VHA-VLB and VHB-VLA or VLA-VHB and VLB-VHA, respectively. Single chain diabodies (scDb) comprise a VHA-VLB and a VHB-VLA fragment which are linked by a linker peptide (P) of 12-20 amino acids, preferably 14 amino acids, (VHA-VLB-P-VHB-VLA). Dual affinity retargeting molecules (“DART” molecules) are diabodies additionally stabilized through a C-terminal disulfide bridge (Moore et al., 2011).

The target cells are any suitable type of cell which can be brought into apoptosis, necrosis or cell lysis by specific interaction with the optionally enriched and/or purified immune cell and/or stem cells. In particular, the target cells are any suitable type of cell which can be brought into apoptosis by specific interaction with the optionally enriched and/or purified immune cell and/or stem cells. In a preferred embodiment, the target cells are selected from cells of a cell line and primary cells. Cell lines have the advantage that these cells are or can be standardized, allowing for easier comparison to control or reference values. For example, the commercially available tumor cell line MV4-11 (cell type: acute monocytic leukemia; DSMZ Accession No: ACC 102) was used successfully in the examples. For example, in case the patient to be treated suffers from cancer or tumor disease, a cell line from the same cancer or tumor disease may be used in the methods. For example, the patient suffers from a leukemia, and the target cells used in the methods of the invention are leukemic cells or a leukemia cell line. Also, it is possible to use primary cells as target cells in the methods of the invention. For example, these primary cells may be obtained from the patient or a subject different from the patient, for example from a donor subject. Further, the target cells may be any primary cells obtained from the patient, and may, for example, be tumor cells.

Therefore, in yet another preferred embodiment, the target cells are selected from cells of a cell line and primary cells. In one more preferred embodiment, the cell line is a tumor cell line and/or is a cell line derived from the same tumor type as the tumor from which the patient suffers. In one more preferred embodiment, the primary cells are derived from the patient and/or are tumor cells.

In another embodiment, the present invention relates to a kit or kit-of-parts comprising

(a) at least one binding agent specifically binding to an apoptosis, necrosis or cell lysis biomarker, wherein the binding agent comprises a detectable label, and optionally one or more of the following:

(b) at least one dye for labeling cells,

(c) an isolated cell population comprising target cells.

In another embodiment, the present invention relates to a kit or kit-of-parts comprising

(a) at least one binding agent specifically binding to activated caspase-3, wherein the binding agent comprises a detectable label, and optionally one or more of the following:

(b) at least one dye for labeling cells,

(c) an isolated cell population comprising target cells.

For example, the kit or kit-of-parts may comprise or consist of an antibody specifically binding to activated caspase-3, a dye for labeling cells and an isolated cell population comprising target cells. The kit or kit-of parts of the examples comprises or consists of an antibody specifically binding to activated caspase-3 (such as a anti-activated caspase-3 antibody labelled with a fluorescent dye), cells of a tumor cell line (such as MV4-11), and a fluorescent dye for labelling cells (such as VPD450).

In another embodiment, the present invention relates to the in vitro use of a kit or kit-of-parts comprising

a) at least one binding agent specifically binding to activated caspase-3, wherein the binding agent comprises a detectable label, and optionally one or more of the following:

(b) at least one dye for labeling cells,

(c) an isolated cell population comprising target cells,

(i) for detecting and/or quantifying Graft-versus-Tumor (GvT) activity, effectivity, compound-mediated toxicity and/or immune-cell mediated cytotoxicity of an isolated cell population comprising immune cells and/or stem cells, and/or

(ii) for determining and/or quantifying Graft-versus-Tumor (GvT) activity, effectivity, compound-mediated toxicity and/or immune-cell mediated cytotoxicity of a cell population comprising immune cells and/or stem cells in a patient to whom the cell population was previously administered, and/or

(iii) for monitoring Graft-versus-Tumor (GvT) activity, effectivity, compound-mediated toxicity and/or immune-cell mediated cytotoxicity of a cell population comprising immune cells and/or stem cells in a patient to whom the cell population was previously administered, and/or

(iv) for identifying a patient to whom a cell population comprising immune cells and/or stem cells was previously administered as a patient for which a change in the cancer and/or transplantation supportive therapy is applicable,

(v) in a method of the present invention described herein, or

(vi) for detecting and/or quantifying Graft-versus-Tumor (GvT) activity, compound-mediated toxicity, effectivity and/or immune-cell mediated cytotoxicity of an isolated cell population comprising immune cells and/or stem cells which is used in the manufacture of a graft and/or pharmaceutical composition for administration to a patient in need thereof.

In another embodiment, the present invention relates to the in vitro use of a kit or kit-of-parts comprising

(a) at least one binding agent specifically binding to an apoptosis, necrosis or cell lysis biomarker, wherein the binding agent comprises a detectable label, and optionally one or more of the following:

(b) at least one dye for labeling cells,

(c) an isolated cell population comprising target cells,

(i) for detecting and/or quantifying Graft-versus-Tumor (GvT) activity, effectivity, compound-mediated toxicity and/or immune-cell mediated cytotoxicity of an isolated cell population comprising immune cells and/or stem cells, and/or

(ii) for determining and/or quantifying Graft-versus-Tumor (GvT) activity, effectivity, compound-mediated toxicity and/or immune-cell mediated cytotoxicity of a cell population comprising immune cells and/or stem cells in a patient to whom the cell population was previously administered, and/or

(iii) for monitoring Graft-versus-Tumor (GvT) activity, effectivity, compound-mediated toxicity and/or immune-cell mediated cytotoxicity of a cell population comprising immune cells and/or stem cells in a patient to whom the cell population was previously administered, and/or

(iv) for identifying a patient to whom a cell population comprising immune cells and/or stem cells was previously administered as a patient for whom a change in the cancer and/or transplantation supportive therapy is applicable,

(v) in a method of the present invention described herein, or

(vi) for detecting and/or quantifying Graft-versus-Tumor (GvT) activity, compound-mediated toxicity, effectivity and/or immune-cell mediated cytotoxicity of an isolated cell population comprising immune cells and/or stem cells which is used in the manufacture of a graft and/or pharmaceutical composition for administration to a patient in need thereof.

For the kit or kit-of-parts and its uses, the same preferred embodiments apply as for the present methods of the invention described herein.

In a preferred embodiment, the binding agent specifically binding to the biomarker, wherein the binding agent comprises a detectable label is a receptor for the biomarker or an antibody to the biomarker, more preferably wherein the biomarker is an activated caspase. In a particularly preferred embodiment, the biomarker is activated caspase-3. In another preferred embodiment, the detectable label is a fluorescent label.

In yet another preferred embodiment, the isolated cell population comprising target cells is an isolated cell population comprising or consisting of a cell line. Such cell lines can be standardized. In a particularly preferred embodiment, the isolated cell population comprising target cells is an isolated cell population comprising or consisting of cell line MV4-11.

In the examples, the commercially available VPD450 (BD) was used as dye for staining target cells for flow cytometry and FACS applications for distinguishing target and effector cells. Suitable dyes are well-known to a skilled person and include PKH26, PKH67, Carboxyfluorescein succinimidyl ester (CFSE) (Tario et al., 2011).

Alternatively, the target cells may be distinguished from effector cells by positively staining the effector cells. In yet alternative embodiments, no staining is performed prior to the step of contacting target cells with the isolated cell population or the immune cells and/or stem cells. For example, the target cells and/or effector cells may be identified, or distinguished from each other, by detecting suitable surface markers for the cells of interest, e.g. by flow cytometry.

The kit or kit-of-parts may comprise or consist of components (a) and (b), (a) and (c) or (a), (b) and (c). Components (a), (b) and (c) may be in separate containers or vials.

The present methods are in particular useful for the cell populations termed an Advanced Therapy Medicinal Product (ATMP) pursuant to EU Regulations such as Regulation (EC) 1394/2007 of 2007. Such medicinal products encompass compositions for gene therapy, compositions for somatic-cell therapy, compositions for tissue-engineered therapy, or combinations thereof (Paul-Ehrlich-Institut, 2010). These medicinal products are complex products, which are challenging from a GMP perspective. To ensure quality, effectiveness and safety, robust and reliable methods are desired for clinical practice. Further, present methods are in particular useful for the cell populations, which are used as starting material or intermediate products in the manufacture of such Advanced Therapy Medicinal Product. For example, cell populations may be tested in the methods of the invention, which are undergoing further cultivation and/or differentiation prior to administration to a patient. Further, cell populations may be tested in the methods of the invention to which further active agents and/or carriers are added prior to administration to a patient or which are undergoing further manipulation such as apheresis prior to administration. For example, such cell populations, which are starting materials or intermediate products for final pharmaceutical products intended for administration to a patient, may comprise or be immune cells, tissue, stem cells, serum, or a bodily fluid.

Moreover, the methods of the present invention are in particular useful for cell population comprising immune cells and/or stem cells, which are a cell suspension, as immune cells and/or stems cells can be easily enriched and/or purified from the cell suspension, thereby further simplifying the procedure for clinical application.

Therefore, in one preferred embodiment, the cell population comprising immune cells and/or stem cells is a cell suspension.

Therefore, in another preferred embodiment, said graft and/or pharmaceutical composition is an Advanced Therapy Medicinal Product (ATMP) selected from a composition for gene therapy, a composition for somatic-cell therapy, a composition for tissue-engineered therapy, or a combination thereof (Paul-Ehrlich-Institut, 2010).

Therefore, in yet another preferred embodiment, the cell population comprising immune cells and/or stem cells is used in the manufacture of a graft and/or pharmaceutical composition for administration to a patient in need thereof and is selected from immune cells, tissue, stem cells, serum, and a bodily fluid.

Moreover, the methods of the invention are useful for grafts comprising immune cells and/or stem cells. The use of grafts comprising immune cells and/or stem cells in transplantation is well known in the art. For example, a disease or disorder treatable by transplantation may be selected from the group consisting of acute myeloid leukemia (AML); acute lymphoid leukemia (ALL); chronic myeloid leukemia (CML); myelodysplastic syndrome (MDS)/myeloproliferative syndrome; malign lymphomas, particularly selected from Morbus Hodgkin, high grade Non-Hodgkin Lymphoma (NHL), mantle cell lymphoma (MCL), low malign NHL, chronic lymphatic leukemia (CLL), multiple myeloma; severe aplastic anemia; thalassemia; sickle cell anemia; immunological defects particularly selected from severe combined immunodeficiency (SCID), Wiskott-Aldrich syndrome (WAS), and hemophagocytic lymphohistiocytosis (HLH); inborn errors of metabolism particularly selected from lysosomal storage disorders and disorders of peroxisomal function; autoimmune diseases; rheumatologic diseases; and recidivisms of any of the above. Even more preferably, said disease or disorder is a hematological malignancy especially selected from acute myeloid leukemia (AML); acute lymphoid leukemia (ALL); chronic myeloid leukemia (CML); myelodysplastic syndrome (MDS)/myeloproliferative syndrome; malign lymphomas, particularly selected from Morbus Hodgkin, high grade Non-Hodgkin lymphoma (NHL), mantle cell lymphoma (MCL), low malign Non-Hodgkin lymphoma (NHL), chronic lymphatic leukemia (CLL), multiple myeloma; severe aplastic anemia; thalassemia; and sickle cell anemia (classification of hematological malignancies summarized in (Taylor et al., 2017)).

For example, the disease or disorder may be treated with a graft comprising hematopoetic stem cells. Such grafts are for example useful for treating a hematological malignancy. The methods of the invention may be used for various types of graft comprising immune cells and/or stem cells. For example, an autologous graft, an allogenic graft, a syngenic graft, a haploidentical graft, or a xenograft (Hatzimichael and Tuthill, 2010; Fabricius and Ramanathan, 2016; Koga and Ochiai, 2019). Moreover, the graft may or may not be a genetically modified graft.

Moreover, in cases where the pharmaceutical product or graft comprises stem cells, various types of stem cells may be present. For example, the graft may comprise cells derived from inducible pluripotent stem cells (iPS). Further, the graft may comprise mesenchymal stem cells, hematopoietic stem cells or embryonal stem cells. In one preferred embodiment, the embryonal stem cells are not human embryonal stem cells.

The pluripotent stem cells used herein may be induced pluripotent stem (iPS) cells, commonly abbreviated iPS cells or iPSCs (Rowe and Daley, 2019). With the exception of germ cells, any cell can be used as a starting point for iPSCs. For example, cell types could be keratinocytes, fibroblasts, hematopoietic cells, mesenchymal cells, liver cells, or stomach cells. There is no limitation on the degree of cell differentiation or the age of an animal from which cells are collected; even undifferentiated progenitor cells (including somatic stem cells) and finally differentiated mature cells can be used as sources of somatic cells in the methods disclosed herein. Somatic cells can be reprogrammed to produce iPS cells using methods known to one of skill in the art. Generally, nuclear reprogramming factors are used to produce pluripotent stem cells from a somatic cell. In some embodiments, at least three, or at least four, of Klf4, c-Myc, Oct3/4, Sox2, Nanog, and Lin28 are utilized. In other embodiments, Oct3/4, Sox2, c-Myc and Klf4 are utilized or Oct3/4, Sox2, Nanog, and Lin28. Once derived, iPSCs can be cultured in a medium sufficient to maintain pluripotency (as described e.g. in WO 2019/023396).

Therefore, in yet another preferred embodiment, the cell population comprising immune cells and/or stem cells is a graft or is used in the manufacture of a graft, preferably wherein the graft (i) is a graft comprising hematopoietic stem cells, and/or (ii) is selected from an autologous graft, an allogenic graft, a syngenic graft, a haploidentical graft, a genetically modified graft and a xenograft.

Therefore, in yet another preferred embodiment, the stem cells are selected from cells derived from inducible pluripotent stem cells (iPS), mesenchymal stem cells, hematopoietic stem cells and embryonal stem cells.

Moreover, the cell population comprising immune cells and/or stem cells may comprise T cells, macrophages and/or NK cells. Such cell populations are in particular useful for adoptive immune cell transfer (reviewed in (Maus et al., 2014)), such as for adoptive T cell transfer, adoptive NK cell transfer or adoptive macrophage cell transfer. Further, cell populations are in particular useful for immune cell-based therapy, such as for T cell-based therapy, NK cell-based therapy or macrophage-based therapy.

Therefore, in yet a further preferred embodiment of the methods of the present invention, the immune cells of the cell population comprise T cells, macrophages and/or NK cells. More preferably, the immune cells of the cell population consist of T cells, macrophages and/or NK cells.

Therefore, in yet a further preferred embodiment of the methods of the present invention, the cell population comprising immune cells and/or stem cells is an immune cell population for adoptive immune cell transfer and/or for immune cell-based therapy.

The cell population of immune cells can be obtained from a subject in need of therapy or suffering from a disease associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the cell population comprising immune cells is obtained from a donor distinct from the subject, the donor is preferably allogeneic, provided the cells obtained are subject-compatible in that they can be introduced into the subject. Allogeneic donor cells may or may not be human-leukocyte-antigen (HLA)-compatible.

In one preferred embodiment, the immune cells comprise or are T cells. Several basic approaches for the derivation, activation and expansion of functional anti-tumor effector cells have been described in the last two decades. These include: autologous cells, such as tumor-infiltrating lymphocytes (TILs); T cells activated ex vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TOR); and non-tumor-specific autologous or allogeneic cells genetically reprogrammed or “redirected” to express tumor-reactive TCR or chimeric TCR molecules displaying antibody-like tumor recognition capacity known as “T-bodies” (Eshhar, 2008; Liu and Guo, 2018). These approaches have given rise to numerous protocols for T cell preparation and immunization. The resulting starting materials comprising cells, intermediate products comprising cells and final pharmaceutical products and grafts comprising cells can be analyzed by the methods of the invention, by detecting, quantifying and monitoring the Graft-versus-Tumor (GvT) activity, compound-medicated toxicity, effectivity and/or immune-cell mediated cytotoxicity of an isolated cell population comprising immune cells and/or stem cells.

The T cells may be derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the subtypes and subpopulations of T cells (e.g., CD4⁺ and/or CD8⁺ T cells) are naïve T (TN) cells, effector T cells (TEFF), memory T cells and subtypes thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and gamma/delta T cells (Golubovskaya and Wu, 2016; Koay et al., 2018; Liu and Guo, 2018).

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

For examples, the method of the present invention was successfully used with different T cell subtypes expressing or not expressing a marker on T cells.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into subpopulations by positive or negative selection for markers expressed or expressed to a relatively higher degree on one or more naive, memory, and/or effector T cell subpopulations.

In some embodiments, the T cells are autologous T cells. For example, for tumor treatment, tumor samples are obtained from patients and a single cell suspension is obtained. The single cell suspension can be obtained in any suitable manner, e.g., mechanically or enzymatically, e.g. using collagenase or DNase. Single-cell suspensions of tumor enzymatic digests are cultured in interleukin-2 (IL-2). The cultured T cells can be pooled and rapidly expanded. Rapid expansion provides an increase in the number of antigen-specific T-cells (as described e.g. in WO 2019/023396).

The autologous T cells can be modified to express a T cell growth factor that promotes the growth and activation of the autologous T cells. Suitable T cell growth factors include, for example, interleukin (IL)-2, IL-7, IL-15, and IL-12 (Paul and Seder, 1994).

In some embodiments, the immune cells are natural killer (NK) cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus (Abel et al., 2018). NK cells can be detected by specific surface markers, such as CD16 and CD56 in humans (as described e.g. in WO 2019/023396).

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Particularly, umbilical CB is used to derive NK cells (Sarvaria et al., 2017).

Of particular interest for adoptive immune cell transfer and/or for immune cell-based therapy for cancer treatment are cytotoxic immune cells, preferably comprising CTLs, CD4⁺ T cells (Restifo et al., 2012), macrophages (Mantovani et al., 2017) and/or NK cells (Fang et al., 2017). Immune cells of the cell population specifically bind to a tumor antigen, thereby exerting a cytotoxic effect on cancer cells expressing said antigen in vivo.

Accordingly, in yet a further embodiment, the immune cell population for adoptive immune cell transfer and/or for immune cell-based therapy comprises or consists of cytotoxic immune cells, preferably comprising CTLs, CD4⁺ T cells, macrophages and/or NK cells, wherein at least one immune cell of the population specifically binds to a tumor antigen.

Various methods exist in the art for providing such immune cells, which specifically bind to an antigen of interest, such as a tumor antigen. For example, natural immune cells, in particular autologous tumor infiltrating lymphocytes (TILs) may be used. Alternatively, immune cells comprising an ex vivo genetically engineered immune cell receptor may be used. Examples are T cells comprising an ex vivo genetically engineered T cell receptor (TCR) (Restifo et al., 2012), NK cells comprising an ex vivo genetically engineered NK cell receptor (Fang et al., 2017) or macrophages (Morrissey et al., 2018) comprising an ex vivo genetically engineered macrophage cell receptor. Such immune cell receptor may for example be a Chimeric Antigen Receptor (CAR) with known antigen-specificity. Further, the immune cell population preferably comprises or consists of CAR-immune cells, preferably wherein the CAR-immune cells comprise or consist of T cells, NK cells and macrophages. For example, the cell population may comprise or consist of CAR-T cells, CAR-NK cells and/or CAR macrophages. Moreover, the ex vivo genetically engineered immune cell receptor may comprise at least one cell-type specific Chimeric Antigen Receptor or a functionally active part thereof. The immune cells of the cell population comprising T cells, NK cells and/or macrophages may be primary immune cells and/or cells of a cell line.

In a yet a further preferred embodiment of the present invention, the immune cells in the immune cell population for adoptive immune cell transfer and/or for immune cell-based therapy comprise or consist of natural immune cells, in particular autologous tumor infiltrating lymphocytes (TILs).

In a yet a further preferred embodiment of the present invention, the immune cells comprise an ex vivo genetically engineered immune cell receptor, such as a T cell receptor (TCR), preferably wherein the ex vivo genetically engineered immune cell receptor is an immune cell receptor with known antigen-specificity or is a Chimeric Antigen Receptor (CAR) with known antigen-specificity.

In a yet a further preferred embodiment of the present invention, the immune cells in the immune cell population comprise or consist of CAR-immune cells, preferably wherein the CAR-immune cells comprise T cells, NK cells and macrophages, and/or the ex vivo genetically engineered immune cell receptor comprises at least one cell-type specific Chimeric Antigen Receptor or a functionally active part thereof.

In a yet a further preferred embodiment of the present invention, the immune cells comprise T cells, NK cells and macrophages, which are primary immune cells and/or cells of a cell line.

The CARs may have any antigenic specificity useful in the treatment of a disease or disorder. The CARs may comprise antigenic specificity for more than one antigen, such as two antigens. Exemplary tumor antigens are known in the art and include CD19, CD20, CD123, mesothelin, CD4, CD5, CD38, CD47, CLL-1, CD33, CD200, CS1, BAFF-R, ROR-1, CD99, HSP70, or BCMA. Among the antigens targeted by the genetically engineered antigen receptors are those expressed in the context of a disease, condition, or cell type to be targeted e.g. via adoptive immune cell transfer. The CAR may contain an extracellular antigen-recognition domain that specifically binds to an antigen. The antigen is preferably a protein expressed on the surface of cells. For example, the CAR may be a TCR-like CAR and the antigen is a processed peptide antigen, such as a peptide antigen of an intracellular protein, which, like a TCR, is recognized on the cell surface in the context of a major histocompatibility complex (MHC) molecule. The chimeric antigen receptor preferably comprises: a) an intracellular signaling domain, b) a hinge and transmembrane domain, and c) an extracellular domain comprising an antigen binding region. Genetically engineered antigen receptors include chimeric antigen receptors (CARs), including activating or stimulatory CARs, costimulatory CARs, and/or inhibitory CARs (iCARs). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone (see e.g. WO 2019/023396 and reviewed in (Dluczek et al., 2019; Kloess et al., 2019)).

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR may include in its extracellular portion one or more antigen binding molecules, such as one or more antigen-binding fragment, domain, or portion, or one or more antibody variable domains, and/or antibody molecules. For example, the CAR may include an antigen-binding portion or portions of an antibody molecule, such as a single-chain antibody fragment (scFv) derived from the variable heavy (VH) and variable light (VL) chains of a monoclonal antibody (mAb).

The terms “tumor-associated antigen,” “tumor antigen” and “cancer cell antigen” are used interchangeably herein. In each case, the terms refer to proteins, or carbohydrates that are specifically or preferentially expressed by cancer cells.

“Treating” or treatment of a disease or condition refers to executing a protocol, which may include administering one or more drugs to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “effective” as used herein, means adequate to accomplish a desired, expected, or intended result. “Effective amount”,“Therapeutically effective amount” or “pharmaceutically effective amount” when used in the context of treating a patient or subject with a compound means that the amount of the compound which, when administered to a subject or patient for treating or preventing a disease, is an amount sufficient to effect such treatment or prevention of the disease.

“Treatment” or “treating” includes (1) inhibiting a disease in a subject or patient experiencing or displaying the pathology or symptomatology of the disease (e.g. , arresting further development of the pathology and/or symptomatology), (2) ameliorating a disease in a subject or patient that is experiencing or displaying the pathology or symptomatology of the disease (e.g. , reversing the pathology and/or symptomatology), and/or (3) effecting any measurable decrease in a disease or symptom thereof in a subject or patient that is experiencing or displaying the pathology or symptomatology of the disease.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites.

Examples of carriers include: liposomes, microspheres (e.g. made of poly(lactic-co-glycolic) acid), albumin microspheres, synthetic polymers, nanofibers, protein-DNA complexes, protein conjugates, erythrocytes, virosomes, and dendrimers.

The term “chimeric antigen receptors” or “CARs”, as used herein, may refer to artificial T cell receptors, chimeric T cell receptors, or chimeric immunoreceptors, for example, and encompass engineered receptors that graft an artificial specificity onto a particular immune effector cell. CARs may be employed to impart the specificity of a monoclonal antibody onto an effector cell, e.g. immune cells, preferably immune cells selected from T cells NK, cells and macrophages, thereby allowing a large number of specific effector cells to be generated, for example, for use in adoptive cell therapy. In specific embodiments, CARs direct specificity of the cell to a tumor associated antigen, for example.

The term “culturing” or “cultivation” refers to the in vitro maintenance, differentiation, and/or propagation of cells in suitable media.

An “anti-cancer” agent or “anti-tumor agent” is capable of negatively affecting a cancer cell/tumor in a patient, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject or patient with cancer.

Moreover, the method of the present invention can be used as a test system for obtaining or not obtaining approval for administration, such as grafting, to a patient in need thereof. The isolated cell population may be tested according to the method of the invention. Depending on the results, the cell population may or may not obtain approval for administration to the patient. In this context, approval or non-approval may depend on the cell population in question and in particular, whether the immune-cell mediated cytotoxicity is a desired or undesired property of the pharmaceutical composition or graft comprising an isolated cell population when administered to a patient. For example, for a graft in tumor disease or adoptive immune cell transfer in a tumor disease, GvT effectivity and/or immune-cell mediated cytotoxicity is typically a desired property of the cell population. In such cases, approval for administration to a patient will typically be provided in case the method of the invention is performed and the amount and/or concentration of the biomarker is above a control, reference or cut-off value. The test system may also be applied for starting cell populations or intermediate cell population products used in the manufacture of a final cell population product, such as a pharmaceutical composition or graft, intended for administration to a patient.

Thus, in a yet a further preferred embodiment of the in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells of the present invention, the immune-cell mediated cytotoxicity is a desired property of the isolated cell population, and: wherein an amount and/or concentration of said biomarker above a control, reference or cut-off value is indicative of a cell population obtaining approval for administration to a patient in need thereof, or for the manufacture of a graft and/or pharmaceutical composition for administration to a patient in need thereof, and wherein an amount and/or concentration of said biomarker below a control, reference or cut-off value is indicative of a cell population not obtaining approval for administration to a patient in need thereof, or for the manufacture of a graft and/or pharmaceutical composition for administration to a patient in need thereof.

Thus, in a yet further preferred embodiment of the in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells of the present invention, the immune-cell mediated cytotoxicity is a desired property of the isolated cell population, and:

wherein an amount and/or concentration of said activated caspase-3 above a control, reference or cut-off value is indicative of a cell population obtaining approval for administration to a patient in need thereof, or for the manufacture of a graft and/or pharmaceutical composition for administration to a patient in need thereof, and wherein an amount and/or concentration of said activated caspase-3 below a control, reference or cut-off value is indicative of a cell population not obtaining approval for administration to a patient in need thereof, or for the manufacture of a graft and/or pharmaceutical composition for administration to a patient in need thereof.

In another preferred embodiment, the immune-cell mediated cytotoxicity is an undesired property of the cell population. In such embodiment, approval for administration may be provided in case the method of the invention is performed and the amount and/or concentration of the biomarker is below a control, reference or cut-off value. The test system may also be applied for starting cell populations or intermediate cell population products used in the manufacture of a final cell population product, such as a pharmaceutical composition or graft, intended for administration to a patient.

Thus, in a yet a further preferred embodiment of the in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells of the present invention, the immune-cell mediated cytotoxicity is an undesired property of the cell population, and: wherein an amount and/or concentration of said biomarker above a control, reference or cut-off value is indicative of a cell population not obtaining approval for administration to a patient in need thereof, or for the manufacture of a graft and/or pharmaceutical composition for administration to a patient in need thereof, and wherein an amount and/or concentration of said biomarker below a control, reference or cut-off value is indicative of a cell population obtaining approval for administration to a patient in need thereof, or for the manufacture of a graft and/or pharmaceutical composition for administration to a patient in need thereof.

Thus, in a yet a further preferred embodiment of the in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells of the present invention, the immune-cell mediated cytotoxicity is an undesired property of the cell population, and:

wherein an amount and/or concentration of said activated caspase-3 above a control, reference or cut-off value is indicative of a cell population not obtaining approval for administration to a patient in need thereof, or for the manufacture of a graft and/or pharmaceutical composition for administration to a patient in need thereof, and wherein an amount and/or concentration of said activated caspase-3 below a control, reference or cut-off value is indicative of a cell population obtaining approval for administration to a patient in need thereof, or for the manufacture of a graft and/or pharmaceutical composition for administration to a patient in need thereof.

Moreover, the methods of the present invention may be used to adapt and amend supportive therapy regimens for a patient to whom a cell population comprising immune cells and/or stem cells was previously administered, in particular in the treatment of cancer and/or for a patient, which has undergone transplantation.

Therefore, in yet another embodiment, the present invention relates to a second cancer and/or transplantation supportive therapy regimen for use in the cancer and/or transplantation supportive treatment of a patient to whom a cell population comprising immune cells and/or stem cells was previously administered and which further receives a first cancer and/or transplantation supportive therapy regimen, wherein the patient is identified as a patient for which a change in the cancer and/or transplantation supportive therapy is applicable by performing a method of the invention, and wherein the second cancer and/or transplantation supportive therapy regimen results in an increased or decreased immune cell function in the patient as compared to the first cancer and/or transplantation supportive therapy regimen.

In yet another embodiment, the present invention relates to a method for providing a second supportive treatment to a cancer patient and/or a transplantation patient in need thereof to which a cell population comprising immune cells and/or stem cells was previously administered, and wherein the patient receives a first cancer and/or transplantation supportive therapy regimen, comprising the steps of:

(i) performing a method of the invention with a sample obtained from the patient,

(ii) administering a second cancer and/or transplantation supportive therapy regimen to the patient, wherein the second cancer and/or transplantation supportive therapy regimen results in an increased or decreased immune cell function in the patient as compared to the first cancer and/or transplantation supportive therapy regimen.

Depending on the cell population in question and the result of the method of the invention, it may be desirable to administer a second cancer and/or transplantation supportive therapy regimen which results in an increased immune cell function in the patient as compared to the first cancer and/or transplantation supportive therapy regimen, or a second cancer and/or transplantation supportive therapy regimen which results in a decreased immune cell function in the patient as compared to the first cancer and/or transplantation supportive therapy regimen.

For example, for a graft in tumor disease or adoptive immune cell transfer in a tumor disease, GvT effectivity and/or immune-cell mediated cytotoxicity is typically a desired property of the cell population (Falkenburg and Jedema, 2017). For example, a second cancer and/or transplantation supportive therapy regimen, which results in an increased immune cell function in the patient as compared to the first cancer and/or transplantation supportive therapy regimen, may be administered to the patient, in case the method of the invention is performed and the amount and/or concentration of the biomarker is below a control, reference or cut-off value.

Alternatively, an excess GvT effectivity and/or immune-cell mediated cytotoxicity may be undesirable in case of a patient, which has previously undergone transplantation. For example, a second cancer and/or transplantation supportive therapy regimen, which results in an increased immune cell function in the patient as compared to the first cancer and/or transplantation supportive therapy regimen may be administered to the patient, in case the method of the invention is performed and the amount and/or concentration of the biomarker is above a control, reference or cut-off value.

For these embodiments of the present invention, the same embodiments apply as for the methods and uses of the invention described herein.

### FIGURE LEGEND

FIG. 1: Flow cytometric detection of activated caspase-3-positive cells (% of VPD450-positive target cells) after incubation of PBMCs (effector cells, E) with MV4-11 (target cells, T). The E:T ratio was adjusted to 25:1, camptothecin was used in a concentration of 6 μM. Additionally, PBMCs were irradiated with 30 Gy X-ray irradiation. Three biological replicates with three technical replicates are shown. ****: p<0.0001 (unpaired t-Test or Welsch test in the case of different variances).

FIG. 2: Flow cytometric detection of activated caspase-3-positive cells (% of VPD450-positive target cells) for the investigation of the cytotoxicity of different separated cell populations. CD56-positive (CD56⁺) and CD56-negative (CD56⁻) cells were separated and additionally used in a mixture (CD56⁺+CD56⁻), cell concentrations were adapted depending on the composition of isolated PBMCs. Camptothecin was used in a concentration of 6 μM. Three biological replicates with three technical replicates are shown. *: p<0.05; ***: p<0.001; ****: p<0.0001 (unpaired t-Test or Welsch test in the case of different variances).

FIG. 3: Flow cytometric detection of activated caspase-3-positive cells (% of VPD450-positive target cells) for the investigation of the influence of Palixizumab® on the cytotoxicity of PBMCs against MV4-11. The E:T ratio was adjusted to 25:1, camptothecin was used in a concentration of 6 μM. Three biological replicates with each three technical replicates are shown. **: p<0.01; ****: p<0.0001 (unpaired t-Test or Welsch test in the case of different variances).

### EXAMPLES

### Materials and Methods

### Preparation of Cells and Co-Incubation

Leukocytes (effector cells) were separated from human blood via density gradient centrifugation, lysis of erythrocytes or in apheresis machines. Cell density was determined by cell counting in Türk's solution and neubauer counting chamber. In some cases, PBMCs were irradiated with 30 Gy X-ray (SARRP, XStrahl) as a control. Separation of CD56⁺ and CD56⁻ cells was performed with EasySep™ Human NK Cell Isolation Kit (Stemcell) and MACS CD56 MicroBeads (Miltenyi), respectively, according to manufacturer's protocol. Incubation of PBMCs with the anti-CD4-antibody Palixizumab® was carried out for 2 h at room temperature with 10⁷ PBMCs in 50 μl phosphate buffered saline (PBS)+1 mM EDTA+0.5% BSA and 5 μg antibody. Human IgG4 kappa protein (abcam) was used as an isotype control. MV4-11 (target cells) were washed twice (centrifugation 300× g, 5 min, RT) with PBS and stained at 6×10⁶ cells per mL in PBS with 1 μM Violet proliferation Dye 450 (BD Horizon™) for 15 min at 37° C. After two washing steps with PBS and complete medium (RPMI 1640 with heat inactivated 10% fetal bovine serum (FBS, gibco)), the target cells were adjusted to 0.5×105 cells per mL in complete medium. Effector cells were adjusted to 1.25×10⁶/mL (PBMCs, incubated or irradiated PBMCs) or in adapted concentrations dependent on the composition of the isolated PBMCs (CD56⁺ and CD56⁻) in complete medium. Camptothecin (stock concentration 12 μM) was used as apoptosis positive control, complete medium as no apoptosis negative control. 0.5 mL effector cells or controls and 0.5 mL target cells (E:T ratio 25:1) were co-incubated for 64-68 h at 37° C., 5% CO2 in a humidified incubator.

### Flow Cytometry

For flow cytometric analysis, 0.5 mL of the co-culture cell suspension were fixed, permeabilized and washed (Fixation Buffer, Staining Perm Wash Buffer, Biozol) according to manufacturer's protocol. Intracellular staining of activated caspase-3 was performed with 10 μl FITC Rabbit Anti-Active caspase-3 antibody (BD Horizon™). Cells were washed in 2 mL Staining Perm Wash Buffer and Cell Staining Buffer (Biozol), respectively. Flow cytometric analysis was performed with a BD FACSCanto™ II.

### Statistical analysis

All data are presented as mean±standard deviation. Statistical analysis using Student's t-test, Welsch test and graphic presentations were made using GraphPad Prism 6.

### Results

Peripheral blood mononuclear cells (PBMCs) were isolated from human blood by density gradient centrifugation. As a control, a part of these cells were irradiated. Effector cells and target cells were incubated in a ratio of 25:1 for 68 h and stained for activated caspase-3.

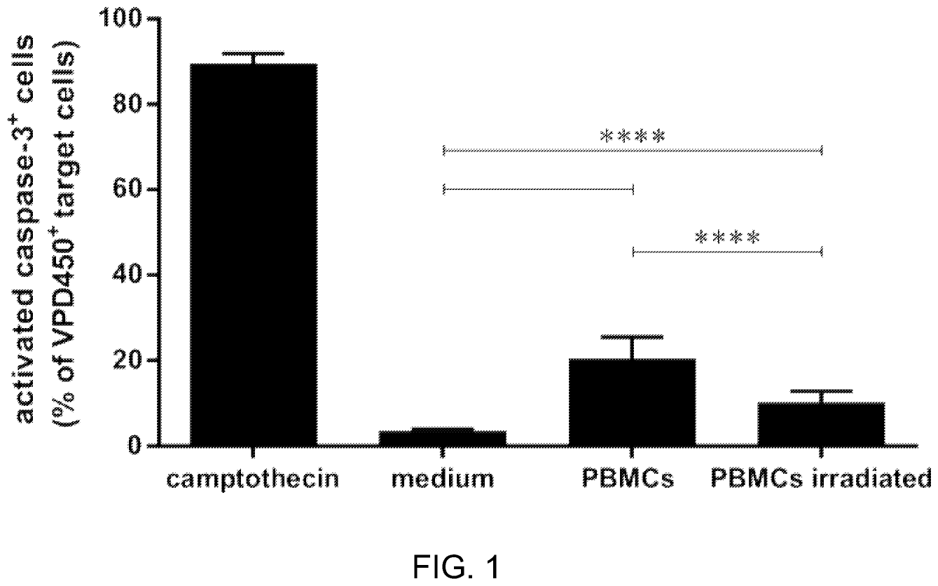

As depicted in FIG. 1, PBMCs are cytotoxic against MV4-11 and this effect is significantly reduced by irradiation with 30 Gy X-ray. To test which type of blood cells is responsible for the cytotoxicity, isolated PBMCs were separated in CD56-positive (CD56⁺, NK cells) and CD56-negative (CD56⁻) cells.

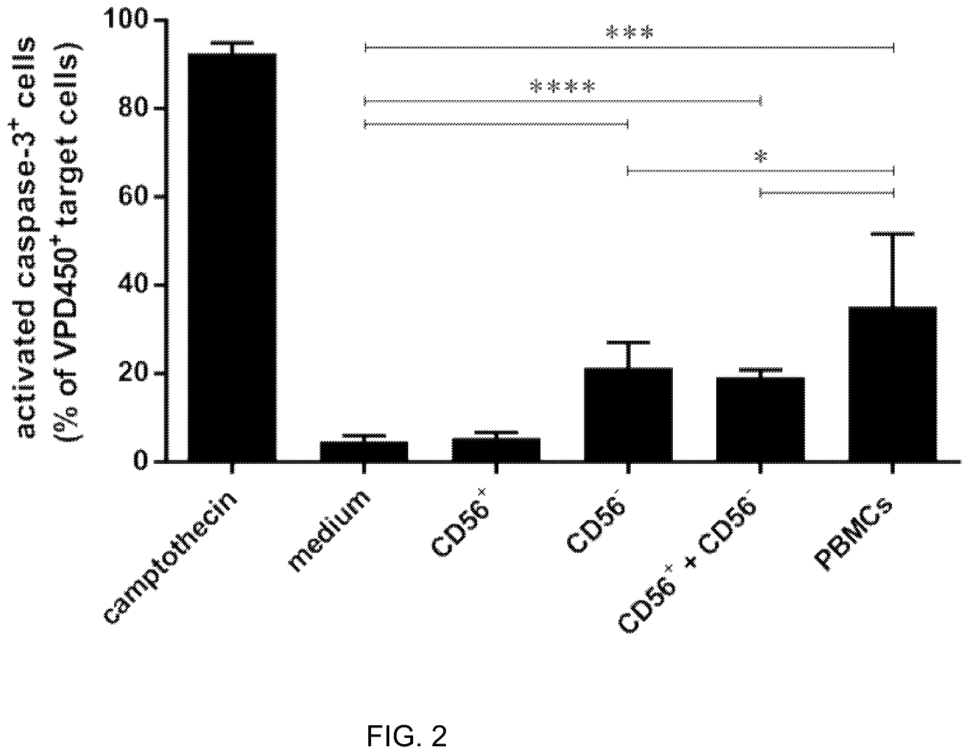

As shown in FIG. 2, CD56⁺ cells are not able to attack MV4-11 cells whereas CD56⁻ cells are as effective against the tumor cells as the mixture of both separated cell types. This demonstrates that the assay quantifies the T cell dependent cytotoxicity.

In further experiments, the applicability of this assay was tested and simulated for an ATMP for the prevention of graft-versus-host disease (GvHD). The ATMP comprises a hematopoietic stem cell transplant that was pre-incubated with the anti-CD4-antibody Palixizumab®. Besides prevention of GvHD, the functionality of the ATMP in terms of anti-cancer capacity (GvL effect) is of high interest. Therefore, it was examined whether the incubation of isolated PBMCs with Palixizumab® influences the cytotoxicity of these cells.

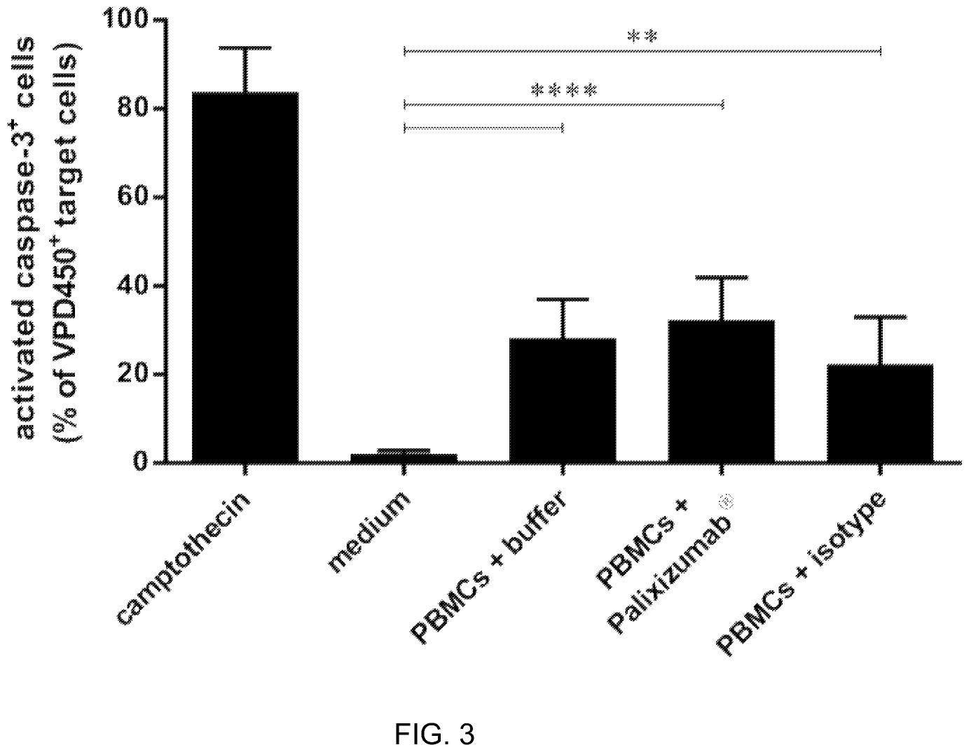

FIG. 3 shows that the incubation of PBMCs with the anti-CD4-antibody Palixizumab® does not influence the cytotoxic effect of these cells. Furthermore these experiments indicate that the assay can be used to quantify the T-cell mediated cytotoxicity of cellular transplants or the influence of supportive therapies.

### CONCLUSION

The aim of this work was the development and testing of a standardized assay for detection and quantification of the cytotoxic effect of cellular transplants or ATMPs, specialized for the use in clinical routine. The T-cell mediated cytotoxicity leads to the apoptosis of target cells and can be measured by flow cytometric detection of activated caspase-3 in target cells. As a direct application, we used PBMCs incubated Palixizumab® and showed that the incubation has no negative effect on the cytotoxicity of PBMCs. Because the detection of apoptosis of the target cells is based on flow cytometry, it is applicable as a clinical relevant standard assay in a conventional diagnostic laboratory.

### REFERENCES

Abel, A. M., Yang, C., Thakar, M. S., and Malarkannan, S. (2018). Natural Killer Cells: Development, Maturation, and Clinical Utilization. Frontiers in immunology 9, 1869. doi: 10.3389/fimmu.2018.01869

Alam, M. K., Brabant, M., Viswas, R. S., Barreto, K., Fonge, H., and Ronald Geyer, C. (2018). A novel synthetic trivalent single chain variable fragment (tri-scFv) construction platform based on the SpyTag/SpyCatcher protein ligase system. BMC biotechnology 18, 55. doi: 10.1186/s12896-018-0466-6

Alwine, J. C., Kemp, D. J., and Stark, G. R. (1977). Method for detection of specific RNAs in agarose gels by transfer to diazobenzyloxymethyl-paper and hybridization with DNA probes. Proceedings of the National Academy of Sciences of the United States of America 74, 5350-5354. doi: 10.1073/pnas.74.12.5350

Amos, P. J., Cagavi Bozkulak, E., and Qyang, Y. (2012). Methods of cell purification: a critical juncture for laboratory research and translational science. Cells, tissues, organs 195, 26-40. doi: 10.1159/000331390

Bader, P., Bornhäuser, M., and Grigoleit, G.-U. (2018). Monitoring, Chimarismusanalysen und Bestimmung der minimalen Resterkrankung (MRD): Allogene Stammzelltransplantation; Leitlinie; Empfehlungen der Fachgesellschaft zur Diagnostik und Thera pie hämatologischer und onkologischer Erkrankungen,
https://www.onkopedia.com/de/onkopedia/guidelines/monitoring-chimaerismusanalysen-und-bestimmung-der-minimalen-resterkrankung-mrd/@@view/pdf/index.pdf

Bader, P., Kreyenberg, H., Henze, G. H. R., Eckert, C., Reising, M., Willasch, A., et al. (2009). Prognostic value of minimal residual disease quantification before allogeneic stem-cell transplantation in relapsed childhood acute lymphoblastic leukemia: the ALL-REZ BFM Study Group. Journal of clinical oncology : official journal of the American Society of Clinical Oncology 27, 377-384. doi: 10.1200/JCO.2008.17.6065

Bishop, M. L., Fody, E. P., and Schoeff, L. E., eds (2013). Clinical chemistry: Principles, techniques, and correlations. Philadelphia, Pa.: Wolter Kluwer/Lippincott Williams & Wilkins.

Blake, D. A., and Ban, B. (2014). “Detection of Metals and Radionuclides Using Rapid, On-site, Antibody-Based Assays,” in Detection of Chemical, Biological, Radiological and Nuclear Agents for the Prevention of Terrorism, ed. J. Banoub (Dordrecht: Springer Netherlands), 195-205.

Bolitho, P., Voskoboinik, I., Trapani, J. A., and Smyth, M. J. (2007). Apoptosis induced by the lymphocyte effector molecule perforin. Current opinion in immunology 19, 339-347. doi: 10.1016/j.coi.2007.04.007

Brunner, K. T., Mauel, J., Cerottini, J. C., and Chapuis, B. (1968). Quantitative assay of the lytic action of immune lymphoid cells on 51-Cr-labelled allogeneic target cells in vitro; inhibition by isoantibody and by drugs. Immunology 14, 181-196.

Buchholz, C. J., Hartmann, J., Schüβler-Lenz, M., and Keller-Stanislawski, B. (2018). CAR-T-Zell-Therapie: Aussichten and Risiken. Dtsch Arztebl International 115, 38-40.

Castella, M., Fernández de Larrea, C., and Martin-Antonio, B. (2018). Immunotherapy: A Novel Era of Promising Treatments for Multiple Myeloma. International journal of molecular sciences 19. doi: 10.3390/ijms19113613

Caulfield, A. J., and Lathem, W. W. (2014). Disruption of fas-fas ligand signaling, apoptosis, and innate immunity by bacterial pathogens. PLoS pathogens 10, e1004252. doi: 10.1371/journal.ppat.1004252

Chowdhury, D., and Lieberman, J. (2008). Death by a thousand cuts: granzyme pathways of programmed cell death. Annual review of immunology 26, 389-420. doi: 10.1146/annurev.immuno1.26.021607.090404

Dagur, P. K., and McCoy, J. P. (2015). Collection, Storage, and Preparation of Human Blood Cells. Current protocols in cytometry 73, 5.1.1-16. doi: 10.1002/0471142956.cy0501s73

Dluczek, S., Tretbar, S., Fricke, S., and Köhl, U. (2019). CAR-T-Zellen: Update 2019. Transfusionsmedizin. doi: 10.1055/a-0833-2631

Duriez, P. J., and Shah, G. M. (1997). Cleavage of poly(ADP-ribose) polymerase: a sensitive parameter to study cell death. Biochemistry and cell biology=Biochimie et biologie cellulaire 75, 337-349.

Engvall, E., and Perlmann, P. (1971). Enzyme-linked immunosorbent assay (ELISA). Quantitative assay of immunoglobulin G. Immunochemistry 8, 871-874.

Eshhar, Z. (2008). The T-body approach: redirecting T cells with antibody specificity. Handbook of experimental pharmacology, 329-342. doi: 10.1007/978-3-540-73259-4_14

Fabricius, W. A., and Ramanathan, M. (2016). Review on Haploidentical Hematopoietic Cell Transplantation in Patients with Hematologic Malignancies. Advances in hematology 2016, 5726132. doi: 10.1155/2016/5726132

Falkenburg, J. H. F., and Jedema, I. (2017). Graft versus tumor effects and why people relapse. Hematology. American Society of Hematology. Education Program 2017, 693-698. doi: 10.1182/asheducation-2017.1.693

Fang, F., Xiao, W., and Tian, Z. (2017). NK cell-based immunotherapy for cancer. Seminars in immunology 31, 37-54. doi: 10.1016/j.smm.2017.07.009

Fink, S. L., and Cookson, B. T. (2005). Apoptosis, pyroptosis, and necrosis: mechanistic description of dead and dying eukaryotic cells. Infection and immunity 73, 1907-1916. doi: 10.1128/IA1.73.4.1907-1916.2005

Forster, R. J., Bertoncello, P., and Keyes, T. E. (2009). Electrogenerated chemiluminescence. Annual review of analytical chemistry (Palo Alto, Calif.) 2, 359-385. doi: 10.1146/annurev-anchem-060908-155305

Fu, Y., Schroeder, T., Zabelina, T., Badbaran, A., Bacher, U., Kobbe, G., et al. (2014). Postallogeneic monitoring with molecular markers detected by pretransplant next-generation or Sanger sequencing predicts clinical relapse in patients with myelodysplastic/myeloproliferative neoplasms. European journal of haematology 92, 189-194. doi: 10.1111/ejh.12223

Ghimire, S., Weber, D., Mavin, E., Wang, X. N., Dickinson, A. M., and Holler, E. (2017). Pathophysiology of GvHD and Other HSCT-Related Major Complications. Frontiers in immunology 8, 79. doi: 10.3389/fimmu.2017.00079

Golubovskaya, V., and Wu, L. (2016). Different Subsets of T Cells, Memory, Effector Functions, and CAR-T Immunotherapy. Cancers 8. doi: 10.3390/cancers8030036

Hatzimichael, E., and Tuthill, M. (2010). Hematopoietic stem cell transplantation. Stem cells and cloning: advances and applications 3, 105-117. doi: 10.2147/SCCAA.S6815

Holliger, P., Prospero, T., and Winter, G. (1993). “Diabodies”: small bivalent and bispecific antibody fragments. Proceedings of the National Academy of Sciences of the United States of America 90, 6444-6448. doi: 10.1073/pnas.90.14.6444

Holtick, U., Chemnitz, J. M., Hallek, M., and Scheid, C. (2015). Allogene Blutstammzelltransplantation-ein Überblick. Klinische Monatsblatter fur Augenheilkunde 232, 641-646. doi: 10.1055/s-0035-1545939

Iliades, P., Kortt, A. A., and Hudson, P. J. (1997). Triabodies: single chain Fv fragments without a linker form trivalent trimers. FEBS Letters 409, 437-441. doi: 10.1016/S0014-5793(97)00475-4

Janeway, C. (2001). Immunobiology: The immune system in health and disease; [animated CD-ROM inside. New York, NY: Garland Publ.

Jerome, K. R., Sloan, D. D., and Aubert, M. (2003). Measurement of CTL-induced cytotoxicity: the caspase 3 assay. Apoptosis: an international journal on programmed cell death 8, 563-571. doi: 10.1023/A:1026123223387

Karimi, M. A., Lee, E., Bachmann, M. H., Salicioni, A. M., Behrens, E. M., Kambayashi, T., et al. (2014). Measuring cytotoxicity by bioluminescence imaging outperforms the standard chromium-51 release assay. PloS one 9, e89357. doi: 10.1371/journal.pone.0089357

Kloess, S., Kretschmer, A., Stahl, L., Fricke, S., and Koehl, U. (2019). CAR-Expressing Natural Killer Cells for Cancer Retargeting. Transfusion medicine and hemotherapy: offizielles Organ der Deutschen Gesellschaft fur Transfusionsmedizin and Immunhamatologie 46, 4-13. doi: 10.1159/000495771

Koay, H.-F., Godfrey, D. I., and Pellicci, D. G. (2018). Development of mucosal-associated invariant T cells. Immunology and cell biology 96, 598-606. doi: 10.1111/imcb.12039

Koga, Y., and Ochiai, A. (2019). Systematic Review of Patient-Derived Xenograft Models for Preclinical Studies of Anti-Cancer Drugs in Solid Tumors. Cells 8. doi: 10.3390/cells8050418

Kröger, N., Badbaran, A., Holler, E., Hahn, J., Kobbe, G., Bornhäuser, M., et al. (2007). Monitoring of the JAK2-V617F mutation by highly sensitive quantitative real-time PCR after allogeneic stem cell transplantation in patients with myelofibrosis. Blood 109, 1316-1321. doi: 10.1182/blood-2006-08-039909

Lakshmipriya, T., Gopinath, S. C. B., and Tang, T.-H. (2016). Biotin-Streptavidin Competition Mediates Sensitive Detection of Biomolecules in Enzyme Linked Immunosorbent Assay. PloS one 11, e0151153. doi: 10.1371/joumal.pone.0151153

Lange, T., Hubmann, M., Burkhardt, R., Franke, G.-N., Cross, M., Scholz, M., et al. (2011). Monitoring of WT1 expression in PB and CD34(+) donor chimerism of BM predicts early relapse in AML and MDS patients after hematopoietic cell transplantation with reduced-intensity conditioning. Leukemia 25, 498-505. doi: 10.1038/Ieu.2010.283

Leitlinienprogramm Onkologie (2017). Supportive Therapie bei onkologischen Patientlnnen: Langversion 1.1, http://leitlinienprogramm-onkologie.de/Supportive-Therapie.95.0.html

Lichtenfels, R., Biddison, W. E., Schulz, H., Vogt, A. B., and Martin, R. (1994). CARE-LASS (calcein-release-assay), an improved fluorescence-based test system to measure cytotoxic T lymphocyte activity. Journal of Immunological Methods 172, 227-239. doi: 10.1016/0022-1759(94)90110-4

Liu, M., and Guo, F. (2018). Recent updates on cancer immunotherapy. Precision clinical medicine 1, 65-74. doi: 10.1093/pcmedi/pby011

Mantovani, A., Marchesi, F., Malesci, A., Laghi, L., and Allavena, P. (2017). Tumour-associated macrophages as treatment targets in oncology. Nature reviews. Clinical oncology 14, 399-416. doi: 10.1038/nrclinonc.2016.217

Maus, M. V., Fraietta, J. A., Levine, B. L., Kalos, M., Zhao, Y., and June, C. H. (2014). Adoptive immunotherapy for cancer or viruses. Annual review of immunology 32, 189-225. doi: 10.1146/annurev-immunol-032713-120136

Mo, Y., Wan, R., and Zhang, Q. (2012). Application of reverse transcription-PCR and real-time PCR in nanotoxicity research. Methods in molecular biology (Clifton, N.J.) 926, 99-112. doi: 10.1007/978-1-62703-002-1_7

Moore, P. A., Zhang, W., Rainey, G. J., Burke, S., Li, H., Huang, L., et al. (2011). Application of dual affinity retargeting molecules to achieve optimal redirected T-cell killing of B-cell lymphoma. Blood 117, 4542-4551. doi: 10.1182/blood-2010-09-306449

Morrissey, M. A., Williamson, A. P., Steinbach, A. M., Roberts, E. W., Kern, N., Headley, M. B., et al. (2018). Chimeric antigen receptors that trigger phagocytosis. eLife 7. doi: 10.7554/eLife.36688

Mullis, K., Faloona, F., Scharf, S., Saiki, R., Horn, G., and Erlich, H. (1986). Specific enzymatic amplification of DNA in vitro: the polymerase chain reaction. Cold Spring Harbor symposia on quantitative biology 51 Pt 1, 263-273.

Muralidharan, S., and Mandrekar, P. (2013). Cellular stress response and innate immune signaling: integrating pathways in host defense and inflammation. Journal of leukocyte biology 94, 1167-1184. doi: 10.1189/jlb.0313153

Olsson, M., and Zhivotovsky, B. (2011). Caspases and cancer. Cell death and differentiation 18, 1441-1449. doi: 10.1038/cdd.2011.30

Paul, W. E., and Seder, R. A. (1994). Lymphocyte responses and cytokines. Cell 76, 241-251. doi: 10.1016/0092-8674(94)90332-8

Paul-Ehrlich-Institut (2010). Arzneimittel für neuartige Therapien (ATMP), https://www.pei.de/DE/arzneimittel/atmp-arzneimittel-fuer-neuartige-therapien/atmp-arzneimittel-fuer-neuartige-therapien-node.html

Petersen, M., and Wengel, J. (2003). LNA: a versatile tool for therapeutics and genomics. Trends in Biotechnology 21, 74-81. doi: 10.1016/S0167-7799(02)00038-0

Pinton, P., Giorgi, C., Siviero, R., Zecchini, E., and Rizzuto, R. (2008). Calcium and apoptosis: ER-mitochondria Ca2+ transfer in the control of apoptosis. Oncogene 27, 6407-6418. doi: 10.1038/onc.2008.308

Prasse, A. A., Zauner, T., Büttner, K., Hoffmann, R., and Zuchner, T. (2014). Improvement of an antibody-enzyme coupling yield by enzyme surface supercharging. BMC biotechnology 14, 88. doi: 10.1186/s12896-014-0088-6

R&D Systems. Detection & Visualization of Antibody Binding, https://www.mdsystems.com/resources/protocols/detection-visualization-antibody-binding

Reed, J. C. (2000). Mechanisms of Apoptosis. The American Journal of Pathology 157, 1415-1430. doi: 10.1016/S0002-9440(10)64779-7

Restifo, N. P., Dudley, M. E., and Rosenberg, S. A. (2012). Adoptive immunotherapy for cancer: harnessing the T cell response. Nature reviews. Immunology 12, 269-281. doi: 10.1038/nri3191

Rissin, D. M., Kan, C. W., Campbell, T. G., Howes, S. C., Fournier, D. R., Song, L., et al. (2010). Single-molecule enzyme-linked immunosorbent assay detects serum proteins at subfemtomolar concentrations. Nature biotechnology 28, 595-599. doi: 10.1038/nbt.1641

Rowe, R. G., and Daley, G. Q. (2019). Induced pluripotent stem cells in disease modelling and drug discovery. Nature reviews. Genetics 20, 377-388. doi: 10.1038/s41576-019-0100-z

Sarvaria, A., Jawdat, D., Madrigal, J. A., and Saudemont, A. (2017). Umbilical Cord Blood Natural Killer Cells, Their Characteristics, and Potential Clinical Applications. Frontiers in immunology 8, 329. doi: 10.3389/fimmu.2017.00329

Sittampalam, G. S., Grossman, A., Brimacombe, K., Arkin, M., Auld, D., Austin, C., et al., eds (2004). Assay Guidance Manual. Bethesda (Md.).

Subklewe, M., Bergwelt-Baildon, M. von, and Humpe, A. (2019). Chimeric Antigen Receptor T Cells: A Race to Revolutionize Cancer Therapy. Transfusion medicine and hemotherapy : offizielles Organ der Deutschen Gesellschaft fur Transfusionsmedizin and Immunhamatologie 46, 15-24. doi: 10.1159/000496870

Tario, J. D., Muirhead, K. A., Pan, D., Munson, M. E., and Wallace, P. K. (2011). Tracking immune cell proliferation and cytotoxic potential using flow cytometry. Methods in molecular biology (Clifton, N.J.) 699, 119-164. doi: 10.1007/978-1-61737-950-5_7

Taylor, J., Xiao, W., and Abdel-Wahab, O. (2017). Diagnosis and classification of hematologic malignancies on the basis of genetics. Blood 130, 410-423. doi: 10.1182/blood-2017-02-734541

ThermoFisher Scientific (2019a). Strategies for Detecting mRNA Northern blotting, Nuclease Protection Assays, In Situ hybridization, and RT-PCR, https://www.thermofisher.com/de/de/home/references/ambion-tech-support/northern-analysis/tech-notes/strategies-for-detecting-mma.html

ThermoFisher Scientific (2019b). Wichtige Informationen zur Real-Time PCR, https://www.thermofisher.com/de/de/home/life-science/per/real-time-per/real-time-per-learning-center/real-time-per-basics/essentials-real-time-per.html

U.S. National Library of Medicine (2019). ClinicalTrial.gov, https://clinicaltrials.gov/ct2/results?cond=&term=chimeric+antigen+receptor&cntr y=&state=&city=&dist=

Vikström S., Lände T. and Blomberg K. DELFIA Cell Cytotoxicity Assays: Technical Tips for the Cell Cytotoxicity Assay using the DELFIA® Technology, https://www.perkinelmer.com/de/lab-products-and-services/application-support-knowledgebase/delfia/delfia-cell-cytotoxicity-assays.html

Wallace, D., Hildesheim, A., and Pinto, L. A. (2004). Comparison of benchtop microplate beta counters with the traditional gamma counting method for measurement of chromium-51 release in cytotoxic assays. Clinical and diagnostic laboratory immunology 11, 255-260. doi: 10.1128/cdli.11.2.255-260.2004

Walter, R. B., Buckley, S. A., Pagel, J. M., Wood, B. L., Storer, B. E., Sandmaier, B. M., et al. (2013). Significance of minimal residual disease before myeloablative allogeneic hematopoietic cell transplantation for AML in first and second complete remission. Blood 122, 1813-1821. doi: 10.1182/blood-2013-06-506725

Wang, J., Nielsen, P. E., Jiang, M., Cai, X., Fernandes, J. R., Grant, D. H., et al. (1997). Mismatch-sensitive hybridization detection by peptide nucleic acids immobilized on a quartz crystal microbalance. Analytical chemistry 69, 5200-5202. doi: 10.1021/ac9706077

Zeiser, R., Wolff, D., Scheid, C., Luft, T., Greinix, H., Dreger, P., et al. (2019). Graft-versus-Host Erkrankung, akut: Leitlinie, Empfehlungen der Fachgesellschaft zur Diagnostik und Therapie hämatologischer und onkologischer Erkrankungen, https://www.onkopedia.com/de/onkopedia/guidelines/graft-versus-host-erkrankung-akut/@@view/pdf/index.pdf

1. An in vitro method for detecting and/or quantifying Graft-versus-Tumor (GvT) activity, compound-mediated toxicity, effectivity and/or immune-cell mediated cytotoxicity of an isolated cell population comprising immune cells and/or stem cells, wherein the isolated cell population is a graft and/or pharmaceutical composition for administration to a patient in need thereof, or is used in the manufacture of said graft and/or pharmaceutical composition, comprising the steps of:
(i) optionally enriching and/or purifying immune cells and/or stem cells from the isolated cell population,
(ii) contacting target cells with the isolated cell population or the immune cells and/or stem cells enriched and/or purified in step (i)
(iii) determining the amount and/or concentration of activated caspase-3 in the target cells of
(ii), wherein an amount and/or concentration of said activated caspase-3 above a control, reference or cut-off value is indicative of Graft-versus-Tumor (GvT) activity, compound-mediated toxicity, effectivity, and/or immune-cell mediated cytotoxicity of the isolated cell population.

2. An in vitro method for determining and/or quantifying Graft-versus-Tumor (GvT) activity, effectivity, compound-mediated toxicity, and/or immune-cell mediated cytotoxicity of a cell population comprising immune cells and/or stem cells in a patient to whom the cell population was previously administered, wherein the cell population is a graft and/or pharmaceutical composition, comprising the steps of:
(0i) providing a sample obtained from the patient,
(i) enriching and/or purifying immune cells from the sample,
(ii) contacting target cells with the immune cells and/or stem cells enriched and/or purified in step (i),
(iii) determining the amount and/or concentration of activated caspase-3 in the target cells of (ii), wherein an amount and/or concentration of said activated caspase-3 above a control, reference or cut-off value is indicative of Graft-versus-Tumor (GvT) activity, effectivity, compound-mediated toxicity and/or immune-cell mediated cytotoxicity of the cell population in the patient.

3. An in vitro method for monitoring Graft-versus-Tumor (GvT) activity effectivity, compound-mediated toxicity and/or immune-cell mediated cytotoxicity of a cell population comprising immune cells and/or stem cells in a patient to whom the cell population was previously administered, wherein the isolated cell population is a graft and/or pharmaceutical composition, comprising the steps of:
(A)
(a) performing the method of claim 1, and
(b) performing the method of claim 2 at 1 or more time point(s) after the cell population was administered to the patient,
or
(B)
(a) optionally performing the method of claim 1, and
(b) performing the method of claim 2 at 2 or more time points after the cell population was administered to the patient,
wherein an increase in amount and/or concentration of said activated caspase-3 in the target cells at a later time point is indicative of an increase in Graft-versus-Tumor (GvT) activity, effectivity, compound-mediated toxicity and/or immune-cell mediated cytotoxicity of the cell population in the patient, and wherein a decrease in amount and/or concentration of said activated caspase-3 in the target cells at a later time point is indicative of a decrease in Graft-versus-Tumor (GvT) activity, effectivity, compound-mediated toxicity, and/or immune-cell mediated cytotoxicity of the cell population in the patient.

4. The in vitro method of claim 2, wherein the patient is treated with at least one cancer and/or transplantation supportive therapy, and wherein the patient is identified as a patient for which a change in the cancer and/or transplantation supportive therapy is applicable
(i) in case an amount and/or concentration of said activated caspase-3 below a control, reference or cut-off value is determined, and/or in case a decrease in amount and/or concentration of said activated caspase-3 in the target cells at least one later time point is determined, or
(ii) in case an amount and/or concentration of said activated caspase-3 above a control, reference or cut-off value is determined, and/or
in case an increase in amount and/or concentration of said activated caspase-3 in the target cells at least one later time point is determined.

5. The method of claim 4, wherein
in case of (i), the change in the cancer and/or transplantation supportive therapy results in an increased immune cell function in the patient, or
in case of (ii), the change in the cancer and/or transplantation supportive therapy results in a decreased immune cell function in the patient.

6. The method of claim 4, wherein the cancer and/or transplantation supportive therapy is administration of one or more immunosuppressant(s) and/or
wherein a change in the cancer and/or transplantation supportive therapy resulting in an increased immune cell function in the patient is administering one or more different immunosuppressant(s) and/or administering a lower dose or frequency of one or more different immunosuppressant(s), or wherein a change in the cancer and/or transplantation supportive therapy resulting in a decreased immune cell function in the patient is administering one or more different immunosuppressant(s) and/or administering a higher dose or frequency of one or more different immunosuppressant(s).

7. An in vitro use of a kit or kit-of-parts comprising
(a) at least one binding agent specifically binding to activated caspase-3, wherein the binding agent comprises a detectable label, and optionally one or more of the following:
(b) at least one dye for labeling cells,
(c) an isolated cell population comprising target cells,
(i) for detecting and/or quantifying Graft-versus-Tumor (GvT) activity, effectivity, compound-mediated toxicity and/or immune-cell mediated cytotoxicity of an isolated cell population comprising immune cells and/or stem cells, and/or
(ii) for determining and/or quantifying Graft-versus-Tumor (GvT) activity, effectivity, compound-mediated toxicity and/or immune-cell mediated cytotoxicity of a cell population comprising immune cells and/or stem cells in a patient to whom the cell population was previously administered, and/or
(iii) for monitoring Graft-versus-Tumor (GvT) activity, effectivity, compound-mediated toxicity and/or immune-cell mediated cytotoxicity of a cell population comprising immune cells and/or stem cells in a patient to whom the cell population was previously administered, and/or
(iv) for identifying a patient to whom a cell population comprising immune cells and/or stem cells was previously administered as a patient for which a change in the cancer and/or transplantation supportive therapy is applicable,
(v) in a method of, or
(vi) for detecting and/or quantifying Graft-versus-Tumor (GvT) activity, compound-mediated toxicity, effectivity and/or immune-cell mediated cytotoxicity of an isolated cell population comprising immune cells and/or stem cells which is used in the manufacture of a graft and/or pharmaceutical composition for administration to a patient in need thereof.

8. The method of claim 1, wherein
(a) the amount and/or concentration of said activated caspase-3 is determined by flow cytometry,
(b) the amount and/or concentration of said activated caspase-3 in step (iii) of claim 1 is determined by flow cytometry and/or comprises contacting the target cells with a binding agent specifically binding to activated caspase-3, wherein the binding agent comprises a detectable label, preferably wherein the proportion of target cells labelled with the binding agent specifically binding to activated caspase-3 is determined, and/or wherein the detectable label is a fluorescent label, and/or wherein the amount and/or concentration of activated caspase-3 is indicative of apoptosis of the target cells, and/or
(c) the target cells are selected from cells of a cell line and primary cells, preferably wherein the cell line is a tumor cell line and/or is a cell line derived from the same tumor type as the tumor from which the patient suffers, or wherein the primary cells are derived from the patient and/or are tumor cells.

9. The method of claim 1, wherein the cell population comprising immune cells and/or stem cells is a cell suspension,
and/or wherein said graft and/or pharmaceutical composition is an Advanced Therapy Medicinal Product (ATMP) selected from a composition for gene therapy, a composition for somatic-cell therapy, a composition for tissue-engineered therapy, or a combination thereof, and/or wherein the cell population comprising immune cells and/or stem cells is used in the manufacture of a graft and/or pharmaceutical composition for administration to a patient in need thereof and is selected from immune cells, tissue, stem cells, serum, and a bodily fluid.

10. The method of claim 1, wherein the cell population comprising immune cells and/or stem cells is a graft or is used in the manufacture of a graft, preferably wherein the graft (i) is a graft comprising hematopoietic stem cells, and/or (ii) is selected from an autologous graft, an allogenic graft, a syngenic graft, a haploidentical graft, a genetically modified graft and a xenograft,
and/or wherein the stem cells are selected from cells derived from inducible pluripotent stem cells (iPS), mesenchymal stem cells, hematopoietic stem cells and embryonal stem cells and/or wherein the immune cells comprise T cells, macrophages and/or NK cells.

11. The method of claim 1, wherein the cell population comprising immune cells and/or stem cells is an immune cell population for adoptive immune cell transfer and/or for immune cell-based therapy.

12. The method of claim 11, wherein the immune cell population for adoptive immune cell transfer and/or for immune cell-based therapy comprises or consists of cytotoxic immune cells, preferably comprising CTLs, CD4+ T cells, macrophages and/or NK cells, wherein at least one immune cell of the population specifically binds to a tumor antigen.

13. The method of claim 11, wherein the immune cells in the immune cell population for adoptive immune cell transfer and/or for immune cell-based therapy comprise or consist of natural immune cells, in particular autologous tumor infiltrating lymphocytes (TILs), or wherein the immune cells comprise an ex vivo genetically engineered immune cell receptor, such as a T cell receptor (TCR), preferably wherein the ex vivo genetically engineered immune cell receptor is an immune cell receptor with known antigen-specificity or is a Chimeric Antigen Receptor (CAR) with known antigen-specificity, more preferably wherein the immune cells in the immune cell population comprise or consist of CAR-immune cells, preferably wherein the CAR-immune cells comprise T cells, NK cells and macrophages, and/or wherein the ex vivo genetically engineered immune cell receptor comprises at least one cell-type specific Chimeric Antigen Receptor or a functionally active part thereof, and/or wherein the immune cells comprise T cells, NK cells and macrophages which are primary immune cells and/or cells of a cell line.

14. The method of claim 1,
wherein the immune-cell mediated cytotoxicity is a desired property of the isolated cell population, and:
wherein an amount and/or concentration of said activated caspase-3 above a control, reference or cut-off value is indicative of a cell population obtaining approval for administration to a patient in need thereof, or for the manufacture of a graft and/or pharmaceutical composition for administration to a patient in need thereof, and wherein an amount and/or concentration of said activated caspase-3 below a control, reference or cut-off value is indicative of a cell population not obtaining approval for administration to a patient in need thereof, or for the manufacture of a graft and/or pharmaceutical composition for administration to a patient in need thereof, or wherein the immune-cell mediated cytotoxicity is an undesired property of the cell population, and:
wherein an amount and/or concentration of said activated caspase-3 above a control, reference or cut-off value is indicative of a cell population not obtaining approval for administration to a patient in need thereof, or for the manufacture of a graft and/or pharmaceutical composition for administration to a patient in need thereof, and wherein an amount and/or concentration of said activated caspase-3 below a control, reference or cut-off value is indicative of a cell population obtaining approval for administration to a patient in need thereof, or for the manufacture of a graft and/or pharmaceutical composition for administration to a patient in need thereof.

15. An in vitro method for detecting and/or quantifying T-cell mediated cytotoxicity of an isolated cell population comprising T cells, wherein the isolated cell population is used in the manufacture of a graft and/or pharmaceutical composition, comprising the steps of:
(i) enriching and/or purifying T cells from the isolated cell population,
(ii) contacting target cells with the T cells enriched and/or purified in step (i)
(iii) determining the amount and/or concentration of activated caspase-3 in the target cells of
(ii), wherein an amount and/or concentration of said activated caspase-3 above a control, reference or cut-off value is indicative of T-cell mediated cytotoxicity of the isolated cell population.

16. The method of claim 15, wherein the method further comprises the step of preparing a graft and/or pharmaceutical composition comprising T cells in case the amount and/or concentration of said activated caspase-3 above a control, reference or cut-off value.

17. The method of claim 16, wherein said step of preparing a graft and/or pharmaceutical composition comprises modifying the T cells to comprise an ex vivo genetically engineered T cell receptor (TCR), preferably wherein the ex vivo genetically engineered T cell receptor is a T cell receptor with known antigen-specificity or is a Chimeric Antigen Receptor (CAR) with known antigen-specificity.

18. The method of claim 15, wherein
(i) step (i) of claim 1 comprises leukapheresis from blood of a patient, and/or
(ii) the graft and/or pharmaceutical composition comprises autologous T cells comprising an ex vivo genetically engineered T cell receptor (TCR), preferably wherein the ex vivo genetically engineered T cell receptor is a T cell receptor with known antigen-specificity or is a Chimeric Antigen Receptor (CAR) with known antigen-specificity.

19. The method of claim 17, wherein the ex vivo genetically engineered T cell receptor has antigen-specificity for a target selected from the group consisting of CD19, CD20, CD123, mesothelin, CD4, CDS, CD38, CD47, CLL-1, CD33, CD200, CS1, BAFF-R, ROR-1, CD99, HSP70, and BCMA.
